(19) United States
(12) Patent Application Publication
(10) Pub. No.: US 2024/0009662 A1
(43) Pub. Date: Jan. 11, 2024

(54) SALIVA COLLECTION APPARATUS AND METHOD

(71) Applicant: National University of Singapore, Singapore (SG)

(72) Inventors: Agata Blasiak, Singapore (SG); Paul Anthony Macary, Singapore (SG); Dean Ho, Singapore (SG)

(21) Appl. No.: 18/025,111

(22) PCT Filed: Aug. 19, 2021
(86) PCT No.: PCT/SG2021/050485

§ 371 Date: Mar. 7, 2023

(30) Foreign Application Priority Data
Sep. 8, 2020 (SG) 10202008709X
Dec. 24, 2020 (SG) 10202013039S

(51) Int. Cl. B01L 3/00

(52) U.S. Cl. CPC B01L 3/502; B01L 2300/0681; B01L 2400/0481

(57) ABSTRACT

An apparatus for collecting a saliva sample is described herein. In a described embodiment, the apparatus comprises a filter; and a pressure generator operable to generate pressure to cause the saliva sample to be transferred through the filter, the filter being configured to reduce a viscosity of the saliva sample as the saliva sample is transferred through the filter. A method collecting a saliva sample from a user is also described, among other aspects.

### FIELD AND BACKGROUND

The invention relates to an apparatus and method for collecting saliva, particularly collecting saliva in a form suitable for use with diagnostic applications.

In recent years, saliva has shown great potential to be implemented in many diagnostic applications such as oral cancer, drug testing and the detection of infectious diseases such as HIV and SARS-CoV-19. A proteomic study conducted in 2016 revealed that saliva contains around 5500 different types of proteins and other biomarkers such as immunoglobulins, blood type substances, enzymes, electrolytes and hormones. While the collection of saliva is usually simple, non-invasive and cost-effective, saliva samples can be difficult to process due to its non-Newtonian behavior caused by the presence of particulate matter and mucins. This is particularly marked in oropharyngeal saliva that contains respiratory mucus expelled by coughing. For measurement or detection of respiratory pathogens, this is a significant factor in the utility of diagnostic tests. Saliva samples that are high in viscosity and/or are very non-uniform may result in measurement difficulties which will lead to inaccurate measurement of biomarkers and consequently cause misdiagnosis.

Freezing of saliva over a time period, precipitation of mucins with chemicals and adjustment of pH are all methods that have been implemented to reduce the viscosity of saliva. These methods, however, require specialized equipment, sample processing conducted by trained personnel, a long processing time and may affect the biomolecular composition of the saliva sample.

It is desirable to provide an apparatus and method for collecting saliva which addresses a least one of the drawbacks of the prior art and/or to provide the public with a useful choice.

### SUMMARY

In a first aspect, there is provided an apparatus for collecting a saliva sample from a user, the apparatus comprising: a filter and a pressure generator operable to generate pressure to cause the saliva sample to be transferred through the filter, the filter being configured to reduce a viscosity of the saliva sample as the saliva sample is transferred through the filter. By reducing the viscosity of the saliva sample by transferring the saliva through a filter, downstream diagnostic processes are facilitated without the need for expensive equipment or trained medical personnel and without affecting any analyte in the saliva sample.

The pressure generator may further comprise a receptacle; the receptacle being arranged to receive the saliva sample and being compressible to generate pressure to cause the saliva sample to be transferred out of the receptacle and through the filter. This arrangement enables a user to straightforwardly transfer the saliva through the filter, without the need for specialist equipment or training.

The receptacle may comprise a seal operable to seal an upper, shielding portion of the receptacle from a lower portion of the receptacle, the lower portion of the funnel receptacle being operable to undergo manual compression. The seal helps to prevent the saliva sample from being accidentally expelled from the device.

The receptacle may comprise a hydrophobic inner surface to encourage the saliva sample to move towards the filter after donation. The receptacle may be in the form of a funnel and the funnel may have an opening in the range from about 8 cm to about 20 cm. The funnel may comprise a plurality of handles. These features enable easily handling of the device to ensure that the receptacle may be held around the user's mouth helping to prevent the loss of any saliva or the exposure of others in the surrounding environment as well as preventing the saliva sample being exposed to impurities from outside of the device.

The pressure generator may comprise a plunger, equivalently a piston, operable to generate pressure to cause the saliva sample to be transferred through the filter. The plunger may be operable to generate a positive or negative pressure on the saliva sample or it may be operable to generate both alternately, thereby enabling repeated cycles of filtering. The plunger may be operable to be inserted into a rigid receptacle for receiving saliva from a user and further to generate a positive pressure on the saliva sample to drive the saliva sample out of the receptacle though the filter. The plunger may be arranged within a collection vessel and operable to draw the saliva though the filter into the collection vessel. The filter may be incorporated into the plunger.

The pressure generator may comprise a suction device operable to draw air out of a collection vessel. The pressure generator may be manually or automatically operated.

The filter may be configured to reduce a viscosity of the saliva sample. The filter may be configured to reduce the shear viscosity at a shear rate of about 50 s⁻¹ of raw saliva by at least about 20%, in particular by at least about 50%. The filter may be configured to increase the uniformity of the saliva sample. The filter may be configured to decrease a coefficient of variation of the saliva sample. The filter may be configured to reduce a coefficient of variation of raw saliva by at least about 80%. Increasing the uniformity of a saliva sample, which may be measured by a reduction the coefficient of variation of a saliva sample, may result in less variation in downstream testing of the saliva sample.

The filter may comprise one or more of a plurality of channels, a multi-layer metal mesh and a porous substrate.

The filter may comprise a plurality of channels, one or more of the channels having a cross sectional width (i.e. the size of the channel measured perpendicular to the direction of the fluidic path through the channel) in the range from about 0.03 mm to about 3 mm which may enable sufficient shear to be induced on the saliva sample to reduce its viscosity and/or increase its uniformity.

One or more of the channels may have a narrowing cross-section which may enable increased shear in the saliva sample to be induced in the channel. The filter may comprise a surface arranged to receive saliva output from the one or more of the plurality of channels having a narrowing cross-section, thereby helping to enable further induction of shear in the saliva sample.

At least two of the channels may have cross sections of different widths. The filter may comprise a plurality of first channels for receiving the saliva sample, a second channel and a third channel in fluidic connection with the plurality of first channels, the plurality of first channels being fluidically connected to the third channel by the second channel. The second channel may have a narrower cross section in at least one direction than the first and third channels, i.e. the width of the second channel perpendicular to the fluidic path through the channel may be narrower than the width of the first or third channels perpendicular to the fluidic path through those channels. The second channel may form a non-zero angle, for example approximately a right angle with the first and third channels. These channel arrangements may enhance the generation of shear in the saliva sample.

The plurality of first channels may be arranged in a substantially circular configuration or a plurality of substantially circular concentric configurations. The third channel may be arranged substantially centrally in a lower surface of the filter. The filter may comprise two third channels.

One or more of the walls of the second channel may have a textured surface which may enhance the generation of shear in the channel.

The filter may comprise stacked first and second modules, the plurality of first channels being comprised within the first module, the third channel being comprised within the second module and the second channel being formed at an interface between the first and second modules, thereby potentially enabling a flexible filter structure according to requirements. The first and second modules may be connected via a first snap fitting, thereby potentially enabling straightforward assembly. The filter comprising a plurality of alternately stacked first and second modules, which may enable alternate cycles of shearing.

One or more of a further filter and a bioactive substance, which may enable additional functionality to be integrated into the filter. The filter may comprise a shielding portion configured to restrict a direction of flow of saliva output from the filter, which may help prevent saliva from escaping from the apparatus.

The apparatus may further comprise a cap operable to close a collection vessel, the filter being comprised within the cap. The filter may be connected to the cap via a second snap fitting. Use of a cap may enable easy integration of the filter into existing collection vessels. Alternatively, the apparatus may comprise a luer adaptor for connecting the filter to a collection vessel and/or the pressure generator.

A collection vessel for the apparatus may further comprise saliva detection and/or deactivation media, the filter being configured to prevent backflow out of the collection vessel.

In a second aspect, a filter is provided, the filter being configured to reduce the viscosity of a saliva sample transferred through it. The filter may be further configured to reduce a coefficient of variation of the saliva sample transferred through it.

In a third aspect a pressure generator is provided, the pressure generator comprising a receptacle for receiving a saliva sample from a user, the receptacle being manually compressible to generate pressure to cause the saliva sample to be transferred out of the receptacle.

In a fourth aspect, a method of reducing the viscosity of a saliva sample is provided, the method comprising: applying pressure to the saliva sample to cause the saliva sample to be transferred through a filter, the filter being configured to reduce the viscosity of the saliva sample as the saliva sample is transferred through the filter. This method may provide a simple yet effective way of reducing the viscosity of saliva for downstream processing which may also be gentle on the saliva avoid affecting the analyte in the saliva sample. The method may comprise applying negative or positive pressure to the sample. The method may comprise alternately applying negative and positive pressure to the sample to drive it back and forth through the filter. The filter may be further configured to increase the uniformity of the saliva sample, or equivalently reduce a coefficient of variation of the saliva sample. As such, the method may also be a method of increasing the uniformity of the saliva sample or equivalently a method of reducing a coefficient of variation of the saliva sample.

In a fifth aspect, a method of collecting a saliva sample from a user is provided, the method comprising receiving, into a compressible receptacle a saliva sample from the user; manually compressing the compressible receptacle to drive the saliva sample from the compressible receptacle into a collection vessel; and receiving the saliva sample in the collection vessel. This method may enable the recovery of a large proportion of the sample donated by the user without requiring specialist equipment or trained personnel. The manual compression may also generate shear on the sample, thereby reducing its viscosity and/or increasing its uniformity. The method may further comprise manually compressing the receptacle to drive the saliva sample through a filter from the receptacle into the collection vessel. Manually compressing the receptacle may comprise squeezing, rolling or twisting the receptacle or a combination of one or more of these methods.

It is envisaged that features relating to one aspect may be applicable to the other aspects.

### BRIEF DESCRIPTION OF THE DRAWINGS

Exemplary embodiments will now be described with reference to the accompanying drawings, in which:

FIGS. 1a and 1b show side and plan views, respectively of an apparatus for saliva collection according to a preferred embodiment;

FIGS. 2a, 2b and 2c show perspective, plan and side views, respectively of a cap for use with the embodiment of FIG. 1;

FIGS. 3a and 3b show perspective and side views, respectively of a filter for insertion into the cap of FIG. 2;

FIGS. 4a, 4b and 4c show perspective, plan and side views respectively of an upper module of the filter of FIG. 3;

FIGS. 5a, 5b and 5c show perspective, plan and side views respectively of a lower module of the filter of FIG. 3;

FIG. 6 shows a method using the apparatus of FIG. 1;

FIGS. 7, 8a, 8b and 8c show methods of manually compressing the funnel of the apparatus of FIG. 1;

FIGS. 9a, 9b and 9c show perspective, side and exploded views, respectively of a four layered filter for use with the apparatus of FIG. 1;

FIGS. 10a, 10b, 10c and 10d show viscosity measurements for the processing of saliva according to a number of methods, including filtering using the filters of FIGS. 3 and 9;

FIG. 10e shows the channel dimensions for the upper module 301 employed to obtain the viscosity measurements of FIGS. 10b, 10c and 10d,

FIGS. 11a and 11 b show alterative channel arrangements for the upper module of the filters of FIGS. 3 and 9;

FIG. 12 shows an alternative channel arrangement for the lower module of the filters of FIGS. 3 and 9;

FIGS. 13a, 13b and 13c show a textured surface for use in the upper and lower filter modules of FIGS. 4 and 5, respectively;

FIGS. 13d and 13e show alternative textured surfaces for use in the upper and lower filter modules of FIGS. 4 and 5, respectively;

FIGS. 14a, 14b and 14c show variations of the upper and lower filter modules of FIGS. 4 and 5 FIGS. 15a, 15b and 15c show perspective, side and perspective views, respectively of a filter module according to a first alternative embodiment;

FIGS. 16a and 16b show perspective and side views, respectively of a filter module according to a second alternative embodiment;

FIGS. 17a, 17b, 17c, 17d, 17e and 17f show alternative embodiments with a plunger employed as a pressure generator;

FIG. 18 shows an alternative embodiment with a plunger employed as a pressure generator; and

FIG. 19 shows an alternative embodiment with a syringe employed as a pressure generator;

FIG. 20 shows experimental results for sample retention by devices according to FIG. 1;

FIG. 21a shows experimental results for the reduction in the number of food particles in saliva using filters according to FIG. 3 and FIGS. 13a and 13b;

FIG. 21b shows experimental results for the reduction in the size of food particles in saliva using filters according to FIG. 3 and FIGS. 13a and 13b;

FIG. 22 shows experimental results for sample recovery using devices having filters according to FIG. 3 and FIGS. 13a and 13b;

FIG. 23a shows experimental results for the change in protein concentration for a device according to FIG. 1 and filter according to FIG. 3 compared with centrifuging;

FIG. 23b shows experimental results for the change in protein concentration for a device according to FIG. 1 and filters according to FIG. 3 and FIGS. 13a and 13b compared with centrifuging;

FIG. 24 shows experimental results showing the change in the uniformity of a saliva sample using a number of saliva processing techniques, including using the device of FIG. 1; and

FIGS. 25a and 25b show experimental results for the reduction in viscosity, and processed sample uniformity, respectively when a saliva collection medium is included in the collection vessel 107 of the device of FIG. 1.

### DETAILED DESCRIPTION OF PREFERRED EMBODIMENTS

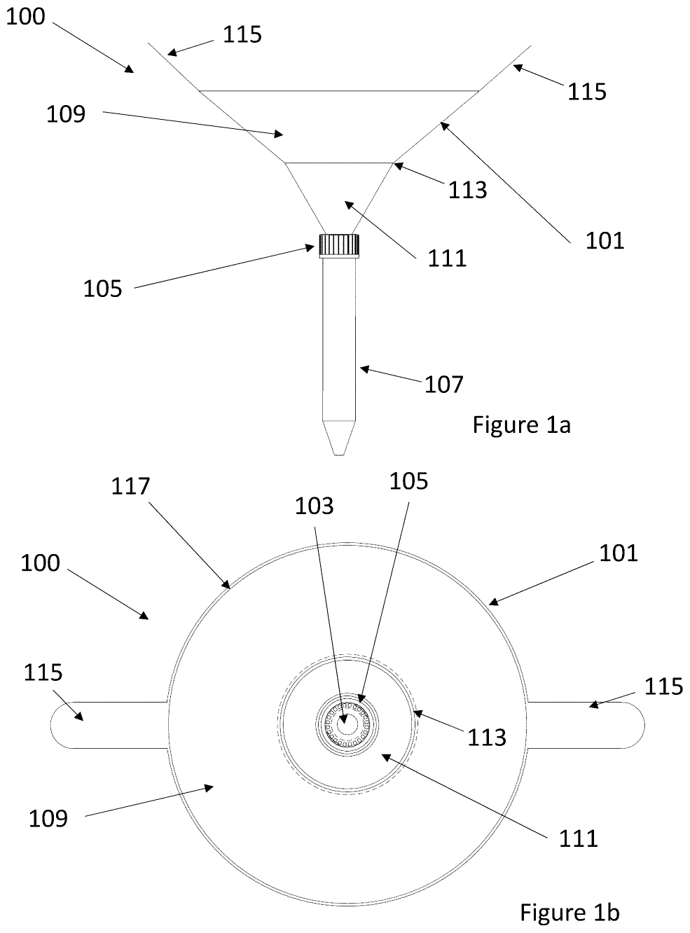

FIGS. 1a and 1b show side and plan views, respectively of a portable saliva collection apparatus 100 according to a preferred embodiment. The apparatus 100 has four main components: a funnel 101 made from a pliable material for receiving saliva, a screw cap 105, a filter 103 located within the screw cap 105, and a collection vessel 107, in this embodiment comprising a tube onto which the screw cap is secured via threads on the neck of the collection tube 107 (not visible). The funnel 101 is in fluidic connection with the collection tube 107 via the filter 103 and is fixed in place at its narrow end between the filter 103 and screw cap 105, such that the funnel 101 is stabilized. As will become evident from the description below, in this embodiment, the funnel 101 functions both as a receptacle for receiving saliva and as a pressure generator to cause the received saliva to be transferred through the filter 103 and into the collection vessel 107. Optionally, saliva detection and/or deactivation media may be included in the collection vessel 107. Examples of saliva detection and/or deactivation media include (but are not limited to) universal transport media, deactivation media, PBS, and saline supplemented with stabilizers and/or enzymes. Collection media may be included in the collection vessel 107 for a variety of different purposes, for example, transport media, etc.

The funnel 101 is divided into two sections 109 and 111 by a seal 113 affixed to the interior wall of the funnel, such as a simple plastic zip for sealing the two walls of the funnel together. The upper, shielding portion 109 has a significantly wider angle than the lower, squeeze portion 111 to facilitate wide opening of the funnel with dimensions sufficient to cover donor's mouth and nose while donating a saliva sample, including oropharyngeal saliva. An example size range for the funnel opening is from about 8 to about 20 cm, similar to the dimensions of a face mask. Examples of the shape of the opening of the funnel include both round and ellipsoidal.

Either the funnel is made from a hydrophobic material or the interior surface of the funnel 101 is coated with a hydrophobic inner coat 117 to encourage saliva to flow down the funnel towards the filter 103 and collection tube 107. The funnel also comprises a pair of handles 115 to assist handling by a user when donating a sample, to hold the funnel 101 open and to shape it such that the portion 109 covers both mouth and nose of the user.

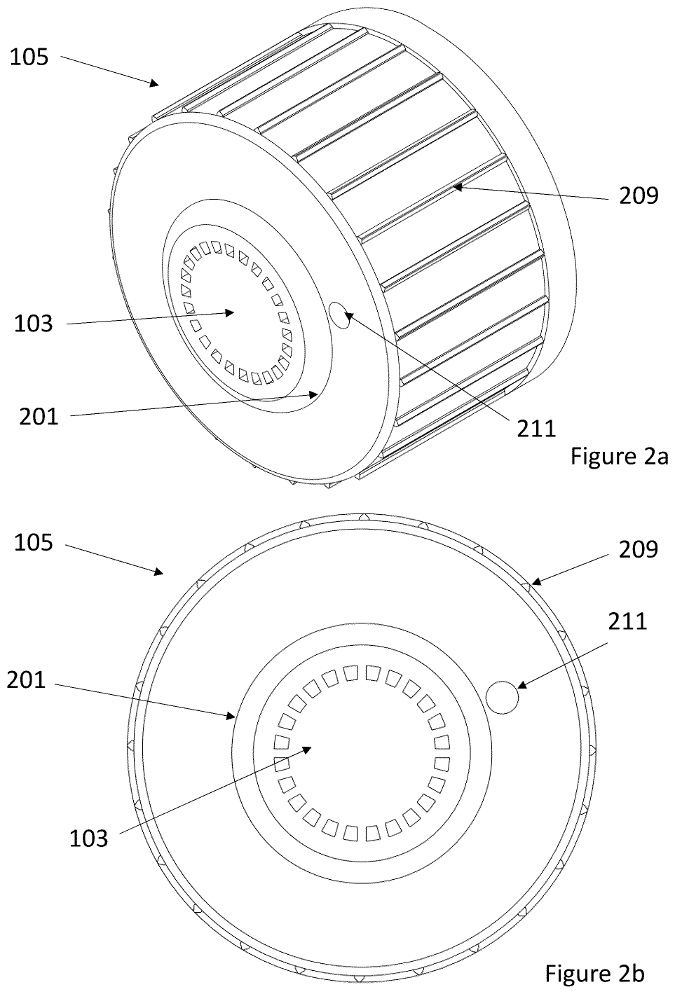

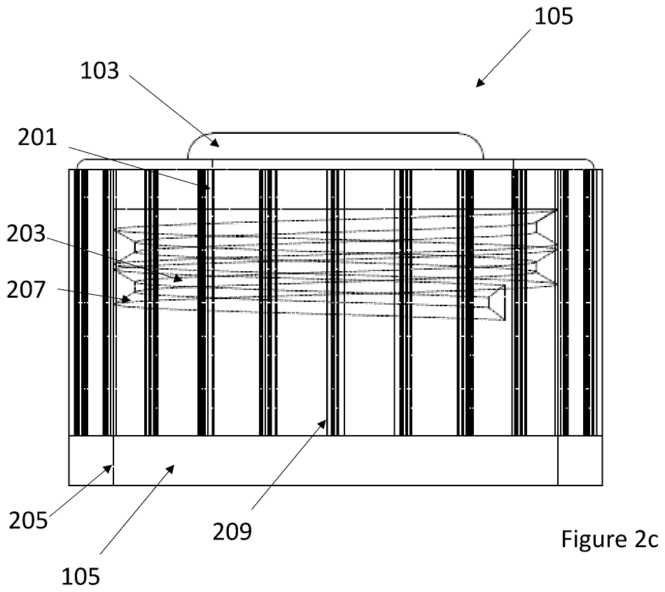

Perspective, plan and side views of the screw cap 105 and filter 103 assembly are shown in FIGS. 2a, 2b and 2c, FIG. 2c also showing the internal structure of the assembly. The cylindrical screw cap 105 is provided with an upper central aperture 201 for receiving the circular filter 103 and a lower aperture, 205, below the filter, for permitting fluid passing though the filter to reach the collection tube 107. The size of the aperture is large enough to receive the filter 103 but smaller than a ridge 505 at the bottom of the filter (see discussion below) enabling the filter 105 to be snap fitted into the aperture. The cap 105 comprises a thread 207 for mating with a thread on the neck of the collection tube 107 and ridges 209 run down the sides of the cap to facilitate screwing onto the collection tube 107. The top of the cap is provided with an air vent 211—an opening connecting inside of the tube with the external environment—to facilitate air release during sample transfer into the collection tube via the filter. The opening of the vent 211 is covered with a microfilter fabric (not visible) to avoid the release of a potentially infectious sample into the environment.

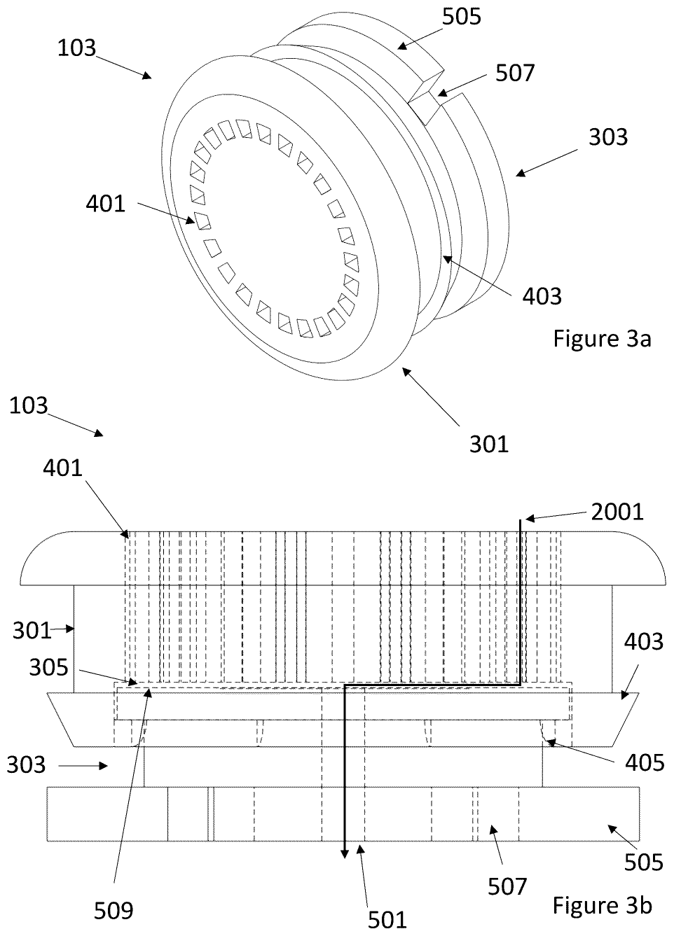

FIGS. 3a and 3b show perspective and side views of the two-module filter 103, the side view of FIG. 3b showing the internal structure of the filter. The filter has a two-piece “stack architecture” comprising upper and lower modules 301 and 303, respectively. Both modules are circular in shape with internal features.

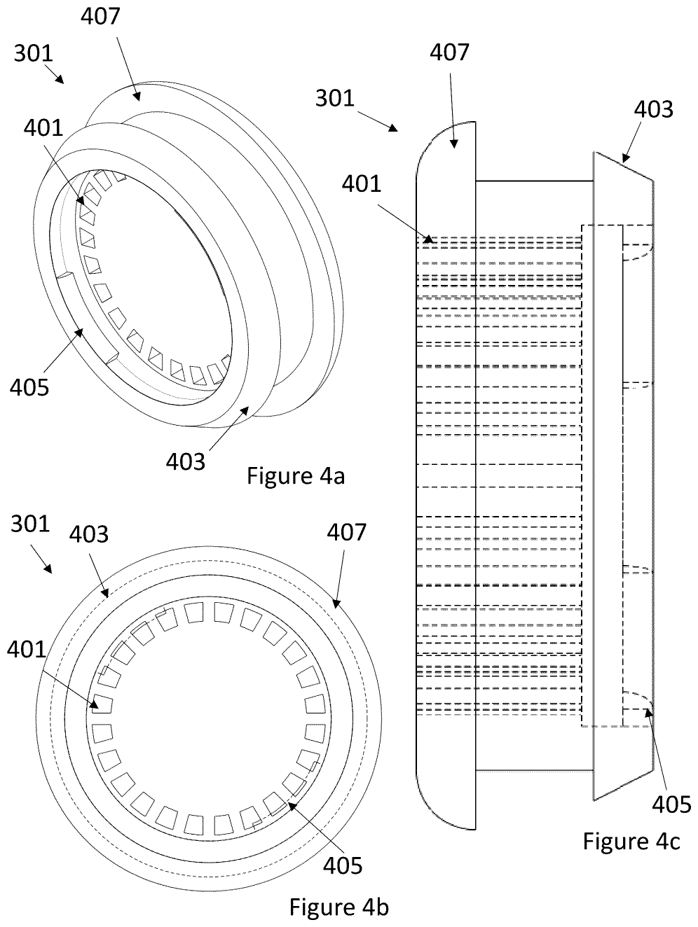

FIG. 4a shows a perspective view (from the underside) of the upper module 301, while FIGS. 4b and 4c show the plan and side views, respectively, the side view of FIG. 4c showing the internal structure of the module. A plurality of channels 401 are arranged in a circular configuration extending from one side of the module to the other for transmitting fluid in the funnel to the lower module of the filter 303, as can be appreciated from FIG. 3b. The upper module 301 comprises a ridge 403 on its underside and projections 405 on the inside of the ridge 403 which enable the upper module 301 to snap on to the lower module 303 of the filter, as can be appreciated from FIG. 3b. The ridge 403 itself enables snap-fitting to the cap.

The upper module of the filter 301, further comprises an overhang 407 which holds the filter in place in the cap 105 and further assists in directing the fluid in the reservoir to enter the collection tube via the channels 401.

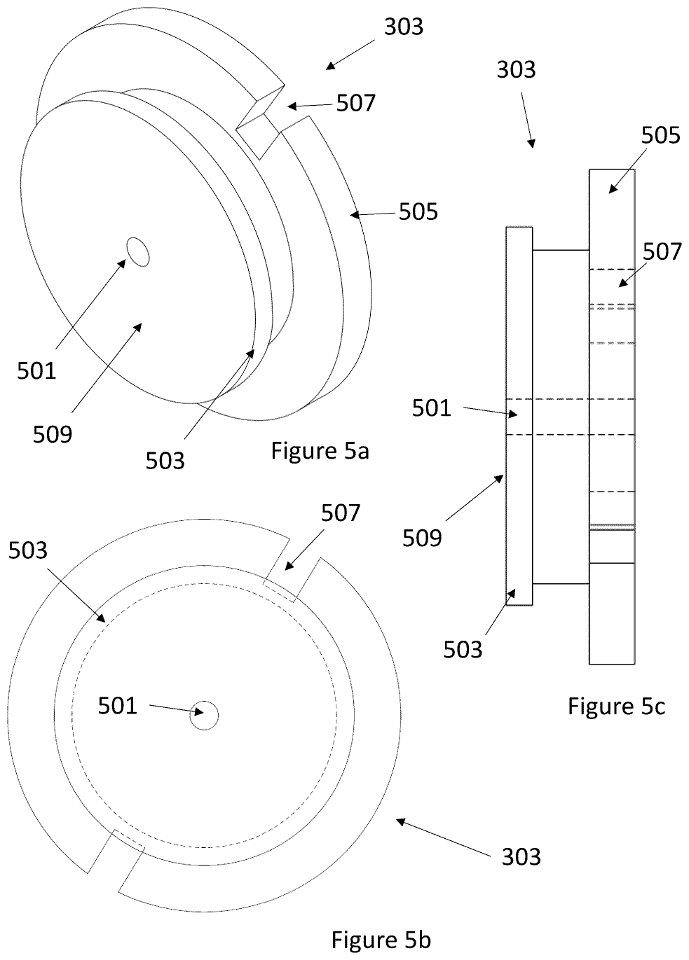

FIG. 5a shows a perspective view of the lower module 303, while FIGS. 5b and 5c show the corresponding plan and side views, respectively, the side view showing the internal structure of the lower module 303. A single circular channel 501 is centrally located in the module and extends through it.

The upper surface 509 of the lower module 303 comprises an overhang 503 which, together with the ridge 403 and projections 405 of the upper module 301 enable the upper module 301 to be snap fitted to the lower module 303. The lower module 303 also comprises a lower extending ridge 505 acting as a shielding portion of the filter for directing a saliva sample passing through the filter downwards and to shield the upper part of the cap and vent. The lower module 303 comprises two optional recesses 507 which may enable the lower module to be held with tweezers and therefore aid assembly.

Returning now to FIG. 3b, which shows the assembled upper 301 and lower 303 modules, it can be seen that a narrow cavity 305 is defined between the upper, multichannel module 301 and the lower, single channel module 303. The single, central channel 501 is aligned with the centre of the circular configuration of the channels 401, i.e. the lower channel 501 is not in direct alignment with any of the upper channels 401.

It can be appreciated that fluidic paths through the filter 103 are defined by the channels 401, the cavity 305 and the channel 501, each forming connecting channels, as shown by arrow 2001.

According to the preferred embodiment, the cavity 305 is narrower than the channels 401. An exemplary range for the width of the channel opening (i.e. at the end of the channel facing the funnel) of channels 401 (in either the radial or circumferential direction) is from about 0.40 mm to about 0.80 mm. An exemplary range for the height of the cavity 305 (perpendicular to the plane of interface between the upper and lower modules) is from about 0.03 mm to about 0.1 mm. An exemplary range for the width of the channel 501 is from about 0.6 to about 0.8 mm.

Preferably, the material forming the funnel 101 is hydrophobic or has a hydrophobic coating; has a high tensile strength; is flexible; is biologically inert so as not to affect the saliva sample; and is non-toxic as it is designed to be placed in contact with human skin during use. The material may be hydrophobic in nature; be coated or partially coated in a hydrophobic coating such as polyethylene or polyurethane; or comprise a hydrophobic layer (whereby an additional coating is not required).

Suitable examples include plastic foil material such as medical grade TPU, food grade plastic, polyethylene, thermoplastic elastomers; polyurethane- or plastic-lined polyester; canvas; cotton; paper; thermoplastic polyurethane, nylon; and silicon.

The filter 103 may be formed by additive manufacturing techniques such as 3-D printing; or moulding. The material forming the filter is preferably inert, rigid, has high tensile strength, and is hydrophobic.

Suitable materials for the filter include acrylics, including various acrylic formulations. In an example, the material may include one or more (meth)acrylic compounds and acrylate based polymers, such as one or more (meth)acrylate monomers, oligomers, and polymers and other acryl based formulations, for example a combination including one or more of: isobornyl acrylate, acrylic monomer, urethane acrylate, epoxy acrylate, and acrylate oligomer. Examples include an acrylic formulation under the product name: VeroClear™ RGD810 of Stratasys Limited.

The cap 105 may be modified from a conventional sample tube screw cap, for example by drilling holes for the vent 211 and for receiving the filter 103, or it may be specially fabricated, for example by moulding or additive manufacturing, such as 3-D printing

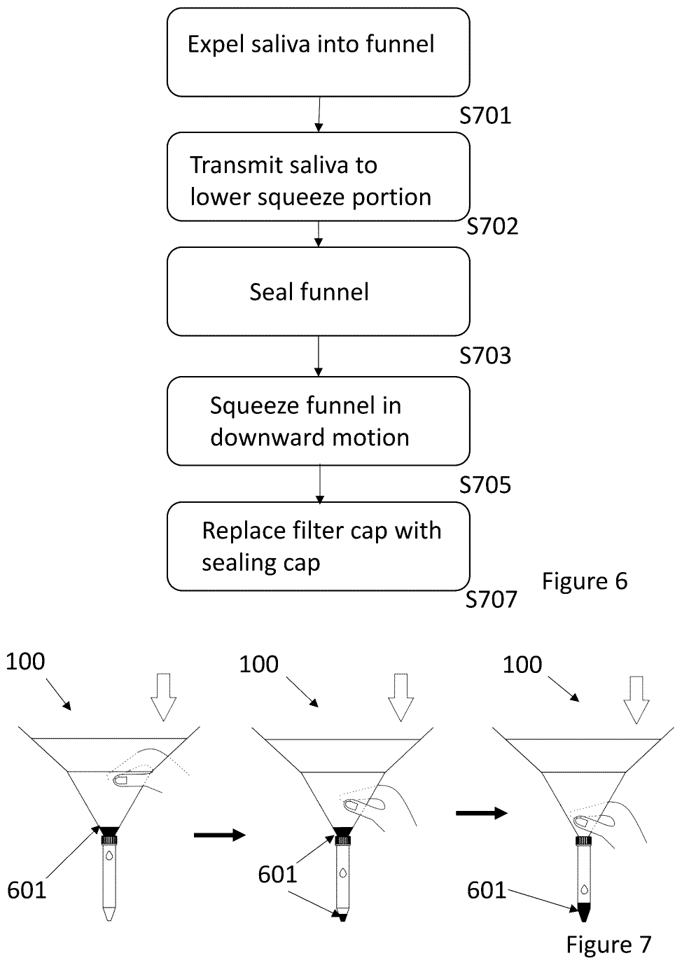

The operation of the device will now be explained in conjunction with FIG. 6, which shows a method of using the apparatus 100 for saliva collection. In step S701, an individual donates a saliva sample 601 by expelling saliva into the funnel. This is done by placing the shielding portion 109 of the funnel 101 around the mouth of the individual by holding the handles 115 either side of the mouth.

In step S702, the individual transmits the sample to the squeeze portion of the funnel 111. The pliable material of the funnel and its hydrophobic coating 117 allows for the downward movement of the saliva into the lower, squeeze portion 111.

In step S703, the funnel is sealed using the seal 113. This prevents the potentially infectious sample 601 from being expelled from the funnel.

In step S705, a downward compressive motion is applied by the user to the squeeze portion 111 to mechanically transfer the saliva through the filter 103 into the collection tube 107, as shown schematically in FIG. 7. The user squeezes the funnel 101 at the squeeze portion 111 between their two fingers and makes downward movement to push the saliva sample 601 through the funnel. The same downward movement can also be made with a clip placed over the funnel.

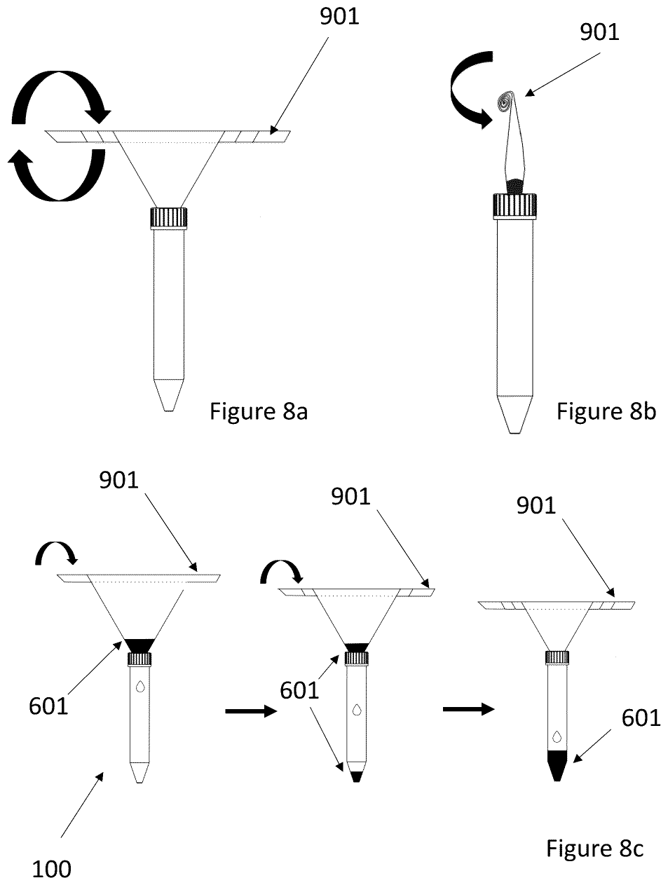

Alternatively, the funnel could be rolled downwards in order to drive the sample through the filter into the collection tube 107. This is shown schematically in FIGS. 8a to 8c. In this method, the top of the funnel 901 is folded over itself using a rotary motion. FIG. 8a shows the partially rolled funnel from front on, while FIG. 8b shows a side view of the partially rolled funnel. The side parts of the rolled funnel 901 can be folded together to further close the opening of the funnel, in a similar way to waterproof “dry bags”, potentially improving its impermeability and aiding to avoid spillage.

FIG. 8c shows the progress of the sample 601 through the apparatus 100 as the funnel 100 is increasingly folded.

For small volume samples, the funnel could be twisted along its vertical axis to create a small pocket above the filter for the sample which can then be squeezed to drive the sample through the filter. This may be particularly employed for samples less than, for example less than about 600 μl, for example where initial samples are split into smaller volumes for generating replicates or performing multiple tests.

Thus, in this embodiment, the funnel is employed both as a receptacle for receiving the saliva sample and as a pressure generator for exerting pressure on the saliva sample to cause it to be transferred it through the filter 103 and into the collection tube 107.

In Step S707, once all of the saliva has been transferred through the filter 103 and collected in the collection tube 107, the cap 105 is removed by unscrewing and replaced with a conventional collection tube screw cap in order to secure the sample in the collection tube 107. The used funnel-cap-filter assembly can then be disposed of in a bio secure manner to avoid the release of potentially infectious substances into the environment.

It will be appreciated from the internal configuration of the filter shown in FIG. 3b that as saliva driven into the filter as described it will first pass through the plurality of channels 401 in the upper module of the filter 301. From there, the fluid will enter the narrow cavity 305 and flow through the cavity 305 to the channel 501 and out of the filter 103, as shown by the arrow 2001.

When the apparatus 100 is employed in this way, the filter helps to remove large particulates from the saliva sample because they are too large to enter the channels 401.

Additionally, the combination of squeezing of the sample in the funnel and the architecture of the filter 103 helps to induce mechanical shear on the saliva. Specifically, the architecture of the filter 103 may ensure that, when the saliva is mechanically transferred through the filter, the sample is pressurized through a series of narrow channels of differing widths. The flow of the saliva against the walls of the channels helps to induce a shear force on the saliva, which may be enhanced on going from a wider channel to a narrower one, for example from channels 401 to the cavity 305. The direction of flow of the saliva also changes abruptly on entering the cavity 305 as the channel 501 is not aligned with any of the channels 401. Saliva flowing out of the channels 401 therefore strikes the upper surface 509 of the lower module 303 under pressure, which may help to induce further shear.

The mechanical shear induced on the saliva sample may cause mucins in the saliva to break up such that a sample of lower viscosity and increased uniformity is received in the collection tube 107.

The filter may reduce the viscosity of raw saliva measured at a shear rate of about 50 s⁻¹ by between from about 20% to about 80%. The filter may reduce the viscosity of raw saliva measured at a shear rate of about 50 s⁻¹ by between from about 50% to about 75%. The measured viscosities may be mean values over a series of measurements taken on respective portions of the same sample.

The mechanical shear induced on the saliva sample may also increase the uniformity of the saliva sample. Uniformity of a saliva sample may be measured by determining a coefficient of variation of the saliva sample. The coefficient of variation is the ratio of the standard deviation to the mean viscosity, expressed as a percentage. The coefficient of variation may be calculated by dividing a sample into various portions and measuring the viscosity of each portion and determining the mean and standard deviation of the measurements.

In a specific example:
i) about 3 ml of raw saliva is collected and processed using the saliva collection apparatus 100;
ii) the about 3 ml of raw saliva is divided into three portions of about 1 ml each;
iii) a first portion of about 1 ml is used for a viscosity measurement and discarded after that measurement; and
iv) step (iii) is repeated for the other two portions.

In total, therefore, measurements of three physical replicates (3×1 ml) of the same saliva sample (3 ml) may be performed, but it is preferred that none of the 1 ml samples go through the measurement twice—rheology measurement destroys the sample. When the above method is performed using a filter according to the preferred embodiment, it is found that the average coefficient of variation of the saliva sample across shear rates is reduced by between from about 80% to about 96%.

Based on the above and to demonstrate the functionality of the apparatus 100, about 3 ml of a saliva sample, including highly viscous posterior oropharyngeal saliva, was collected from a healthy individual. Its physical appearance and physical properties relevant to pipetting process were assessed. The raw saliva sample had non-uniform color and texture. The sample was cloudy, large food contamination and highly viscous fractions were visible. No droplets were formed. The sample was inserted into the saliva collection apparatus 100. After the sample moved downward on the hydrophobic surface 117 of the funnel 101 to the bottom of the squeeze portion 111, the squeeze portion 111 was sealed, and the funnel was rolled as shown in FIG. 8c to induce pressure on the sample to pass through the filter 103. Fizzing at the release side of the filter 103 was visible. The processed sample was more uniform in appearance and the presence of food contamination and highly viscous mucins was limited. The formation of droplets was observed during pipetting. The shear provided by the channel architecture was sufficient to break the mucins and increase the uniformity of the saliva sample.

Low viscosity is desirable for the use of saliva in subsequent diagnosis methods. Saliva is a complex matrix to work with, with the high viscosity affecting sensitivity and specificity of the downstream assays, e.g. PCR, bead immunoassay. Reducing the viscosity of the saliva may also help to improve the ease of its handling.

Uniformity of the saliva sample is also desirable for downstream testing of the sample as it may result in less variation during testing.

The apparatus 100 therefore provides a simple handheld device for collecting and pre-processing a saliva sample for a streamlined downstream detection assay. This sheer that may be generated by the user squeezing the saliva through the filter 103 may result in marked changes to the fluid properties that are conducive to the subsequent fluid transfer to a diagnostic. Without this level of processing, accurate diagnosis may be precluded and/or substantial additional processing may be required. Thus, the apparatus 100 and corresponding method described above may enable saliva samples to be obtained, separated, and preserved without the need for specialized equipment, trained personnel, harsh chemical intervention, freezing and associated delays. The functionality of the apparatus 100 may therefore improve the quality of interface with the downstream assays without using additional pre-processing protocols.

The features of apparatus 100 may also provide a number of advantages in addition to enabling the reduction in the viscosity of the saliva and potential increase in uniformity of the sample.

For example, the filter 103 may also provide a barrier to backflow. Thus, where saliva detection and/or deactivation media is included in the collection vessel 107, this may prevent detection and/or deactivation media entering the funnel and potentially being ingested by the user.

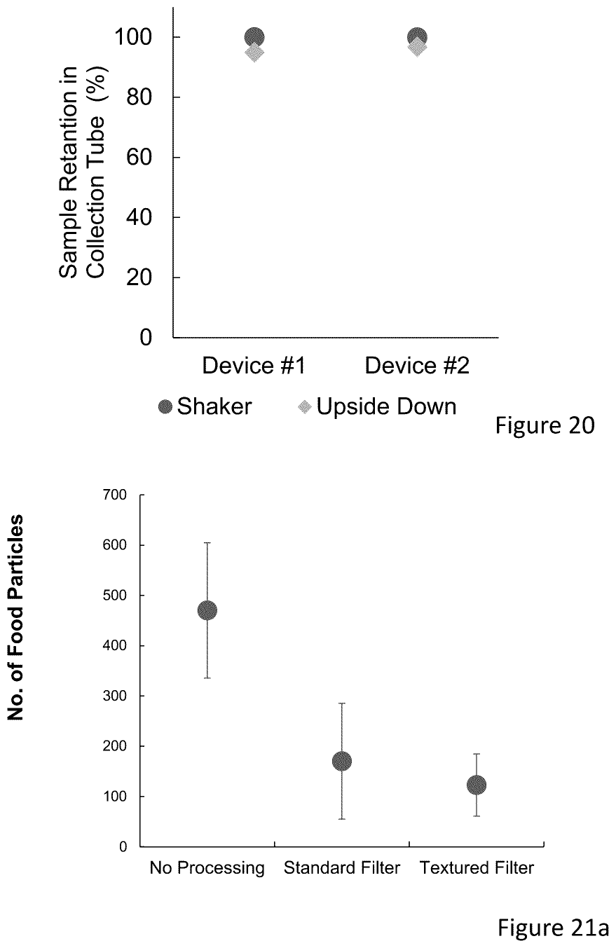

FIG. 20 shows experimental results which demonstrate the prevention of backflow by filters according to the preferred embodiment. In a first experiment 3 ml of MiliQ water was placed in each of two collection vessels 107 before caps 105 with filters 103 and funnels 101 attached according to the preferred embodiment were screwed onto the collection vessels 107. The two devices were kept on a shaker at 100 rpm for 1 h. The experiment was repeated 3 times.

The weight of the collection tube before and after each experimental round were used to calculate sample retention. The results are indicated by the solid circles in FIG. 20 as average±Standard Deviation. A greater than about 99% sample retention was found for both samples.

In a second experiment, two devices were prepared as above but instead of being shaken were immobilized upside down for 1 h. The experiment was repeated 3 times. The weight of the collection tube before and after each experimental round was used to calculate sample retention. The results are indicated by diamonds in FIG. 20. A greater than about 94% sample retention was found for both samples.

The filter 103 may be effective in reducing the number and average size of food particles in the filtered saliva sample. Further, the filter 103 may be able to process a large quantity of food particles before becoming blocked. In order to demonstrate this functionality, chili flakes of known weight were added to the funnel of a device according to the preferred embodiment until it was no longer possible to pass MiliQ water through the filter. By weighting, it was determined that the amount of chili flakes required to block the filter was about 6.5 g.

Further, the pliable character of the funnel 101 may offer multiple advantages:
The pliable, hydrophobic material of the funnel may facilitate the downward movement of the saliva sample.
The funnel opening (shielding portion 109) is wide thereby potentially helping to prevent the spreading of potentially infections particles during sample collection, with individuals in a close vicinity of each other. The shielding portion includes a broader edge enabling the individual to place the broader edge of the shielding portion in an adaptable shape around their mouth and nose, thus assisting in shielding the environment from the exposure to the expelled, potentially infectious sample and also the sample from the environment and helping to reduce the risk of contamination of the sample. Despite the width of the funnel 101, the apparatus 100 may remain compactible for storage and transport as the funnel can be folded.
The shielding portion of the funnel has handles 115 on two sides of its opening that may facilitate easy holding and adjustment while collecting the sample. The pliable nature of the material may enable the adjustment of the shape of the opening to the individual while collecting the sample.
The shielding portion 109 of the funnel 101 may also protect the user when transmitting the sample to the collection tube 107 via the filter 103.
The pliable material of the funnel may allow for incorporating a simple plastic zip to seal the funnel and help prevent release of the saliva into the environment.
The pliable material of the funnel may allow the user to squeeze the funnel, either locally or roll it, and induce pressurized passage through the filter 103 towards: (i) higher sample recovery (ii) mechanical shear and breaking up the mucins, and the functional architecture may help enable fluid sheer while minimising concerns of material rupture.
The pliable material may facilitate easy and universal mounting of the funnel 100 into most caps after modification increasing its compatibility with existing lab tubes and broadening the scope of its use.

High sample recovery even with small samples has been demonstrated experimentally using the twisting technique described above. 546 μl of MiliQ water was placed in the funnel 101 of a device according to FIG. 1. About 86.3% of the sample was recovered after pushing it through by twisting the funnel.

Advantageously, the external part of the filter is architecturally developed such that it snaps onto a collection tube cap that has a hole drilled in the middle of it. This mode of mounting may advantageously help in:
avoiding using adhesives that could potentially contaminate the sample or shorten its shelf life;
discarding the funnel and the filter by simply unscrewing the cap; and
compatibility with existing detection kit tubes.

The device may also enable easy scaling up and manufacturing and may be made using only inert materials, without requiring adhesives.

Thus, the device may assist in sample acquisition with no or a low level of supervision. This device may therefore reduce or eliminate physical contact with medical personnel, as is important for the safety of airborne infectious diseases diagnostics.

The preferred embodiment should not be construed as limitative.

In an example of a variation of the preferred embodiment, the filter 103 may comprise further modules stacked between modules 301 and 303 in order to provide repeated shearing cycles, thereby enabling a further reduction in the viscosity of the saliva and potentially an increase in the uniformity of the sample.

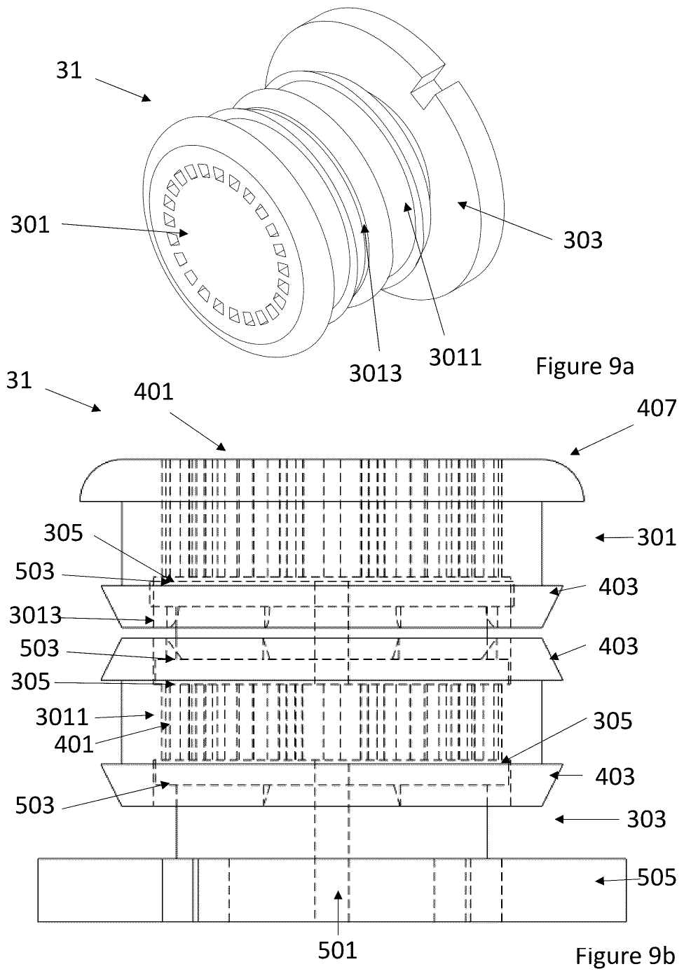

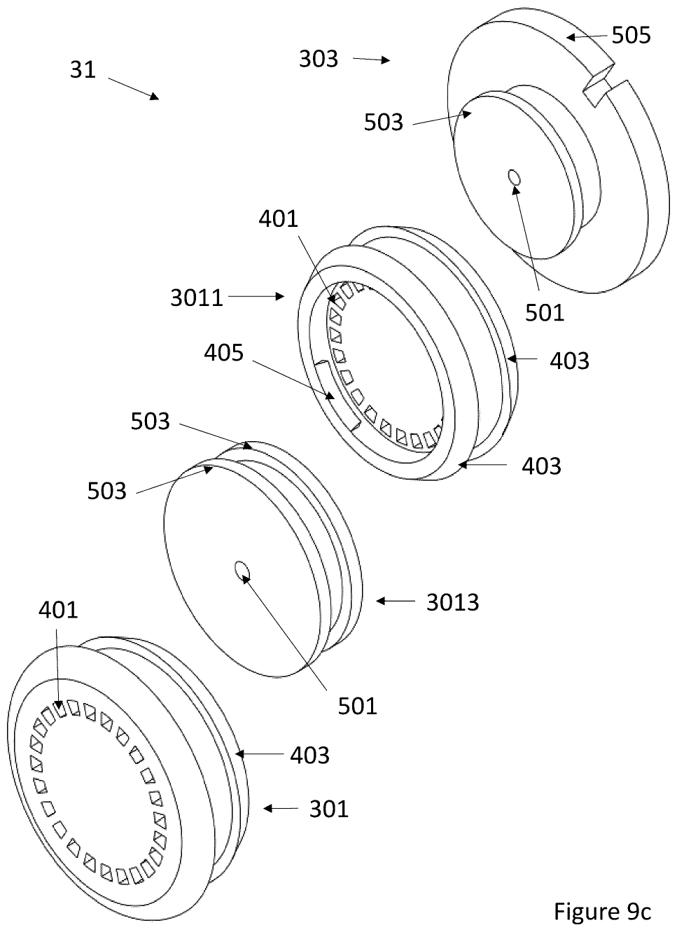

FIGS. 9a, b, c show perspective, side and exploded views of a four-module filter 31, the side view of FIG. 9b showing the internal structure of the filter. The filter has a four-piece stack architecture comprising modules 3013 and 3011 sandwiched between upper and lower modules 301 and 303 as described above.

Module 3013 is similar to the lower module 303, comprising a single, central channel 501. However, module 3013, comprises an overhang 503 on both sides in order to permit snap fitting to both the upper module 301 and module 3011.

Module 3011 is similar to the upper module 301, comprising a plurality of channels 401 arranged in a circular configuration. However, module 3011 comprises a ridge 403 and projections 405 on both sides for snap fitting to the module 3013 and the lower module 303.

From FIG. 9b, it can be observed that when the upper and lower modules are snapped together, three narrow cavities 305 are now defined between the upper, multichannel module 301 and the single channel module 3013; between the single channel module 3013 and the multichannel module 3011; and between the multichannel module 3011 and the lower, single channel module 303. Both central channels 501 are aligned with the centre of the circular configuration of the channels 401 of the multichannel modules 301 and 3011, i.e. the channels 501 are not in direct alignment with any of the channels 401.

Thus, when saliva is transferred through the four-module filter 31, it will be appreciated that two shearing cycles are performed on the saliva; the first by modules 301 and 3013 and the second by modules 3011 and 303. Further modules having the same configuration as modules 3011 and 3013 could be stacked to provide additional shearing cycles.

In order to demonstrate the functionality of the apparatus 100 with both two- and four-module filters, oropharyngeal saliva samples from seven volunteers were subjected to processing using different techniques. The techniques applied were:
i) centrifugation for 10 minutes, at about 2500 g and about 4° C., to obtain supernatant and pellet fractions (about 40% of the bottom volume);
ii) treatment with about 10 mM of DTT;
iii) filtration with a two-module filter 103; and
iv) filtration with a four-module filter 31.

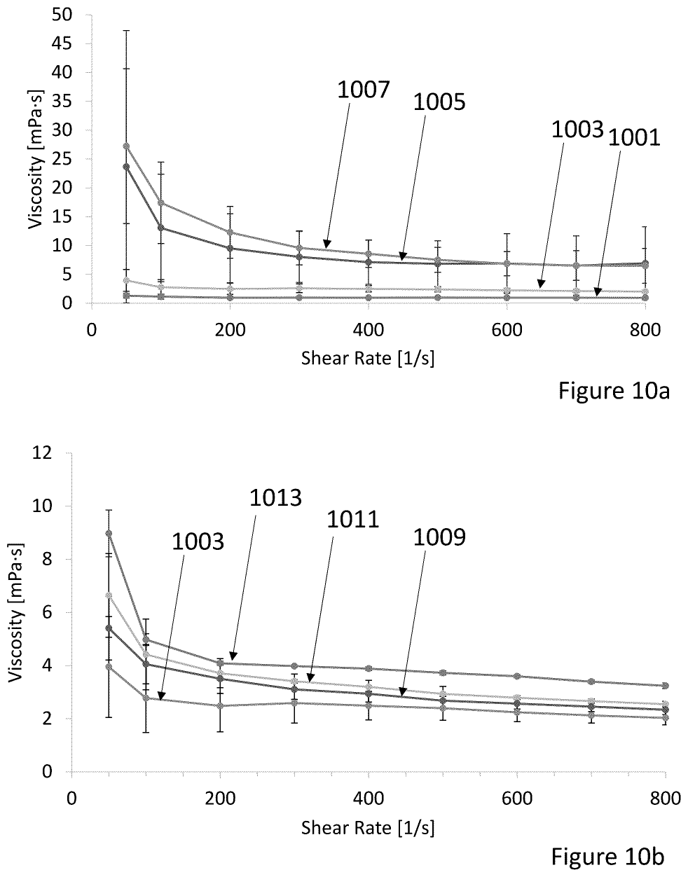

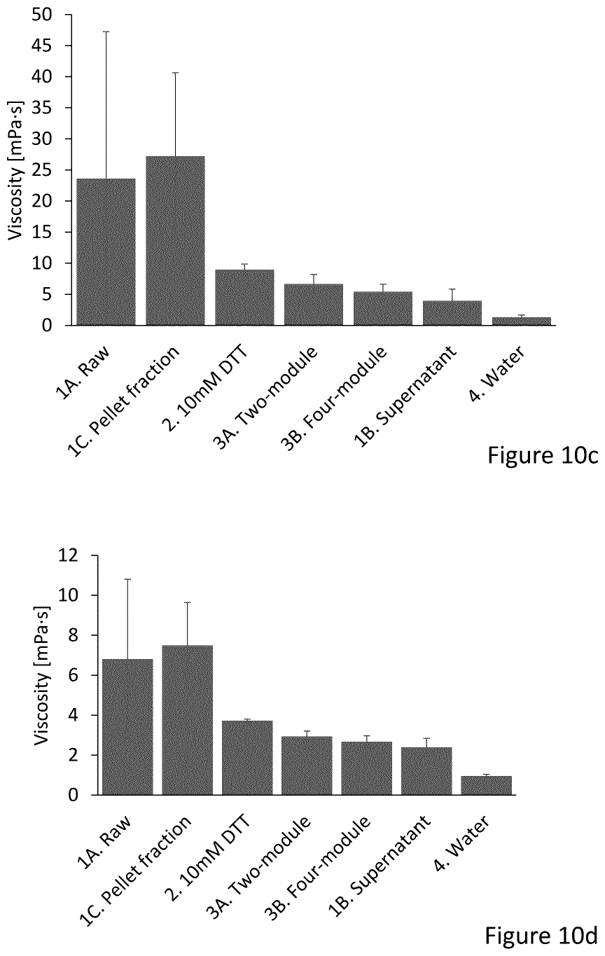

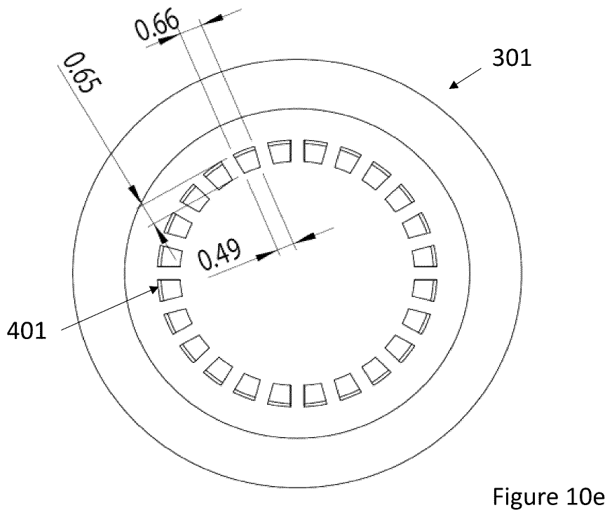

The two- and four module filters had channels 401 of about 0.65 mm in length, with an outer arc width of about 0.66 mm and an inner arc width of about 0.49 mm, as shown in FIG. 10(e). The size of the cavity or cavities 305 was about 0.1 mm and the diameter of channel 501 was about 0.8 mm.

The viscosities of the processed samples were measured with an Anton Paar MCR 302 modular compact rheometer using the cone plate measuring system (CP25-2). The samples (except for the centrifugation supernatant sample) were vortexed briefly for 5 s to get a homogeneous solution for testing. The samples were loaded onto the measuring plate with a disposable pipette and shear rates were varied incrementally from 0 to 800.0 s⁻¹ at 12 different speeds. The measurements were carried out at about 25° C. and viscosity measurement of each sample type was replicated 3 times. The viscosity of water and raw saliva samples were also measured for comparison.

The average saliva viscosity for raw and centrifuged saliva obtained as described above is shown in FIG. 10a. Water is shown by line 1001, the supernatant fraction by line 1003, the raw saliva by line 1005 and the pellet fraction by line 1007.

FIG. 10b shows the average saliva viscosity for processed saliva. As before, the supernatant fraction of the centrifuged sample is shown by line 1003, the four-module filtered sample is shown by line 1009, the two-module filtered sample is shown by line 1011 and the sample subjected to DTT treatment is shown by line 1013.

FIGS. 10c and 10d show the viscosity for all samples at shear rates of about 50 s⁻¹ and about 500 s⁻¹, respectively.

As shown in the figures, the modular filter devices according the preferred embodiment perform better than DTT, decreasing the viscosity of the saliva to a greater extent at all shear rates measured. Both modules achieved a greater than about 50% reduction in the shear viscosity at a shear rate of about 50 s⁻¹ relative to the raw sample. Although the shear viscosity following filtration with the two- and four-module devices is higher than the supernatant fraction following centrifugation, this is to be expected because the filtered saliva includes the pellet fraction. This may contain a higher concentration of the analyte of interest than the supernatant fraction.

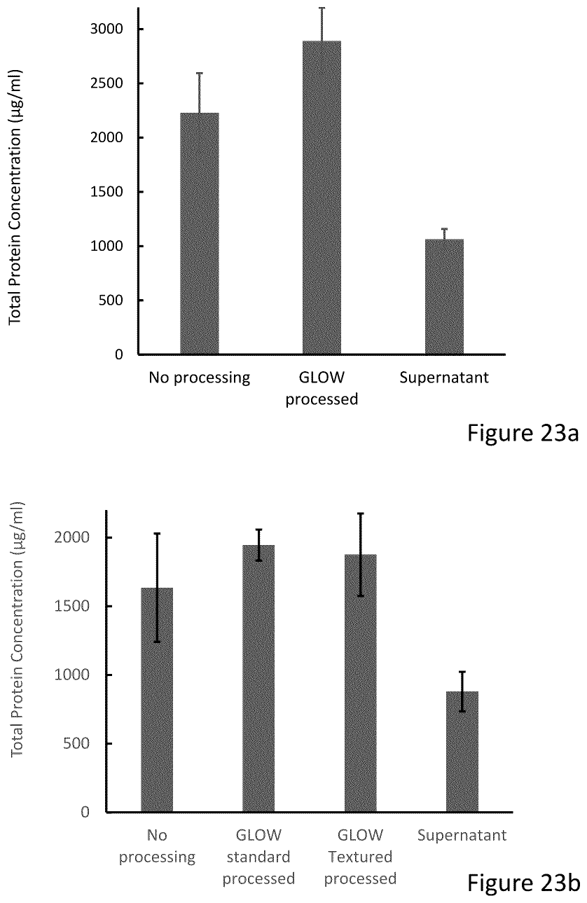

This effect was demonstrated experimentally, the results being shown in FIG. 23a. Back throat saliva was pooled from 12 individuals. The sample was processed through a saliva collection device according to FIG. 1 with a two-module filter according to FIG. 3. The protein content in samples that were unprocessed, processed with the device according to the preferred embodiment (denoted “GLOW processed” in FIG. 23a) and processed by centrifuging (10 min, 300 rpg; denoted “Supernatant” in FIG. 23a) were compared using BCA assay performed as per manufacturer's instructions. This was performed three times for each tested sample. Not only was the recovered protein concentration for the device according to the preferred embodiment much greater than obtained by centrifuging, a slight increase in total protein concentration was detected relative to the unprocessed saliva was also observed.

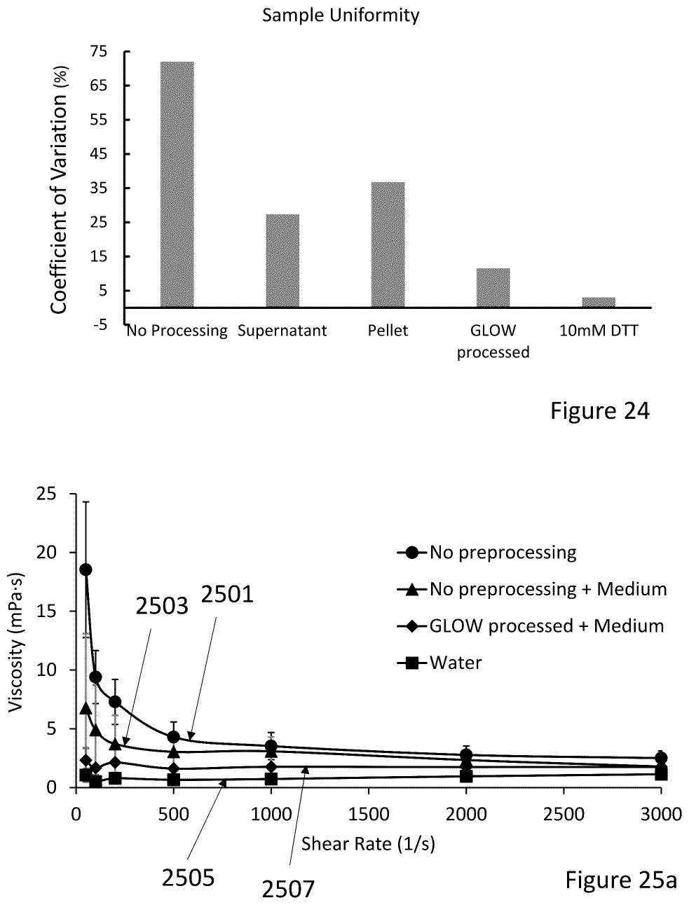

Sample uniformity following processing was also measured for some of the samples of FIGS. 10a and 10b, including the two-module filter (denoted “GLOW processed”) and the results are shown in FIG. 24, with the uniformity expressed as an average coefficient of variation of the viscosity across the shear rates. The coefficient of variation of viscosity for each shear rate was defined as the ratio of the standard deviation of the viscosity to the mean viscosity over three replicated viscosity measurements taken from different portions of the same sample, expressed as a percentage. The coefficient of variation using the two-module filter was decreased by about 6.2 times relative to that of unprocessed saliva, indicating an increase in uniformity caused by the filter.

The modular filter devices according to preferred embodiment thereby enabled both a reduction in viscosity and improvement in uniformity of the saliva while retaining analyte in the sample, enabling accurate diagnostics to be performed on the sample. Further, the technique of mechanical shear stress employed to achieve viscosity reduction and improved uniformity is not as harsh on the potential analyte structure as treatment with DTT.

As will be appreciated from FIG. 10b, the four-module filter (shown by line 1009) shows an incremental viscosity reduction over the two-module filter (shown by line 1011) at low shear rates.

As discussed above, optionally, saliva detection and/or deactivation media may be included in the collection vessel 107. Examples of saliva detection and/or deactivation media include (but are not limited to) universal transport media, deactivation media, PBS, and saline supplemented with stabilizers and/or enzymes. Collection media may be included in the collection vessel 107 for a variety of different purposes, for example, transport media, etc.

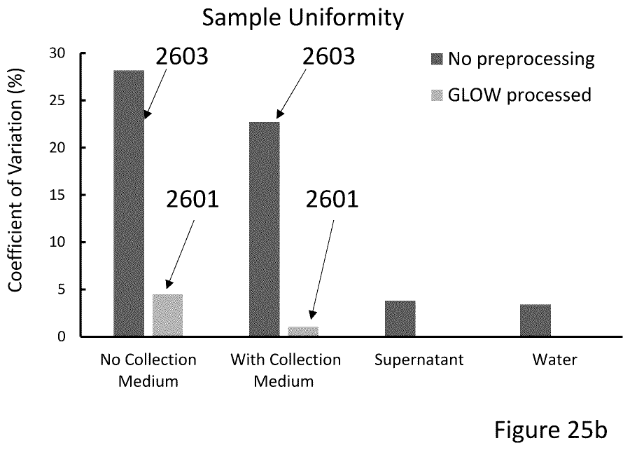

The presence of collection media has been shown experimentally not to negatively impact any reduction in viscosity or increase in sample uniformity achieved by filters according to embodiments. FIG. 25a shows viscosity results for a saliva sample with no pre-processing (line 2501), a saliva sample in the presence of a collection medium but no other pre-processing (line 2503), a saliva sample processed by a device according to FIG. 1 and in the presence of a collection medium (line 2507) and water (line 2505). The results were obtained by pooling back throat saliva from 12 individuals. Collection media constituted 20% volume of the final sample volume (2.5 ml). The incubation time with collection media was 1 h. Three samples were processed for each technique. The processing with the device according to FIG. 1 was shown to decrease viscosity below that achieved by using a collection medium alone. The sample pre-processed using the filter according to FIG. 3 and exposed to the collection media behaved like a Newtonian fluid. The viscosity drop at a shear rate of 50 s⁻¹ due to use of the filter 103 of FIG. 3 when paired with the collection media and without was about 2.8 times and about 2.9 times, respectively.

Sample uniformity was also calculated at shear rates 1000-3000 1/s to avoid incorporating bias from measurement noise for low viscosity samples at low shear rates detected in the water sample. The results are shown in FIG. 25b with samples processed using the filter of FIG. 3 (denoted “GLOW processed” and indicated by line 2601 in FIG. 25b). The coefficient of variation was significantly lower in samples processed with the filter according to the preferred embodiment relative to unprocessed samples (line 2603) with (about 21 times lower) and without (about 6.2 times lower) the collection media and achieved the same levels as supernatant and water samples, indicating a decrease in the coefficient of variation due to the use of the filer.

In further variations of the preferred embodiment, the dimensions and shape of the upper 301 and lower 303 modules (and intermediate modules 3011 and 3013, as appropriate) could be varied according to requirements. For example, the number, location, and size of the channels in the upper 301 and lower 303 modules (and intermediate modules 3011 and 3013, as appropriate) can be varied.

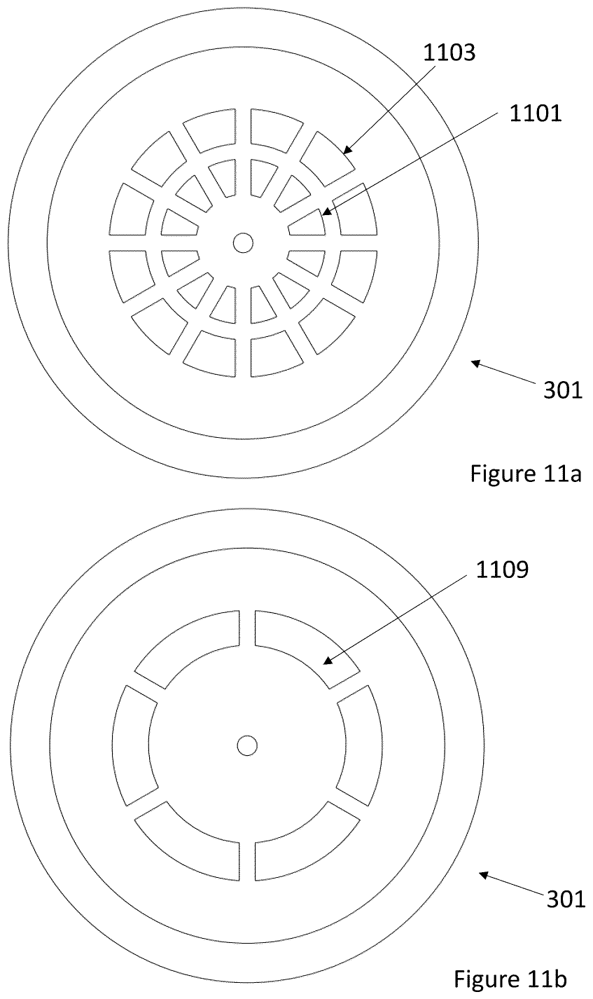

FIGS. 11a and 11 b show examples two alternative arrangements of channels in the upper module 301 (the arrangement of which could also be implemented in an intermediate module 3011 as appropriate). In the upper module 301 of FIG. 11a, the channels 401 are arranged in two concentric circles 1101 and 1103. The channels of the inner circle 1101 are necessarily smaller in cross section that those of the outer circle 1103, thereby providing a higher level of shear.

In the upper module 301 shown in FIG. 11b, the channels 1109 are arranged in a single circular configuration but there are fewer channels forming the circle, each being relatively wider than those of FIG. 4 or 11a.

It will be appreciated that a combination of the channel configurations shown in FIGS. 4, 11a and 11b could be employed. Further, other arrangements and/or channel sizes could be employed.

It will be appreciated that the example channel sizes discussed above in relation to the preferred embodiment are not intended to be limiting and that other channel dimensions could be employed according to requirements. In general, smaller channels will be expected to provide a higher level of shear and a greater reduction in viscosity of the sample and/or increase in the uniformity of the sample. The amount of shear achievable will depend on the size of the channels that can be formed using filter manufacturing techniques while enabling the saliva to be transferred through the filter with the chosen pressure induction, or generation method.

Mid-sized channels may be preferred for less challenging samples, for example, when a pressure that can be applied is limited, when very low viscosity of the processed sample is not critical, or as part of a filter stack comprising multiple layers of modules. Mid-size pores such as those shown in FIG. 11b may provide a lower shearing effect but may not require as much pressure to be exerted in order to cause the sample to be transferred through them. For certain use cases, low pressure may be preferable (for example, for users with lower hand strength). Further, a mid-level of shearing may be adequate for the desired use. For example, if the mechanical shear is being employed together with mild a chemical treatment in order to reduce the viscosity of the sample and/or improve its uniformity.

Preferably, the cross-sectional width of all of the channels lies in the range from about 0.03 mm to about 3 mm. Channels within this range may induce shear and remove food particles from the saliva sample, while enabling saliva to be transferred through the filter with the use of a pressure generator.

Embodiments described herein may therefore provide a flexible design which may be adapted by varying channel sizes or the addition or removal of stacked modules according to use requirements.

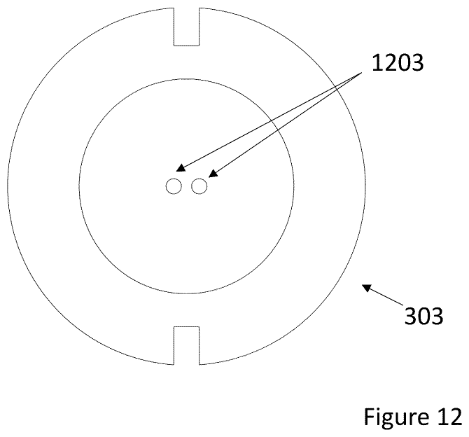

FIG. 12 shows an example of an alternative arrangements of channels for the lower module 303 (the arrangement of which could also be implemented in an intermediate module 3013, as appropriate). In the lower module 303 shown in FIG. 12, two channels 1203 are provided either side of the centre.

Although all of the channels of the preferred embodiment are shown as being uniform in width although their length, some or all of the channels 401 or 501 could instead have a narrowing geometry (narrowing in the direction of flow of saliva), thereby increasing the shear force exerted by them.

Although the upper and lower modules are described as being separable, it will be appreciated that the upper and lower modules could be formed integrally, for example by additive manufacturing methods such as 3-D printing.

Although the upper 301 and lower modules 303 of the filter are shown as having smooth surfaces between channels, the lower surface of the upper module 301 and/or the upper surface of the lower module 303 may be textured (and the corresponding surfaces of modules 3011 and 3013, as appropriate). This is to increase the shear generated as the saliva passes through the narrow cavity 305 due to turning and squeezing through the saliva sample.

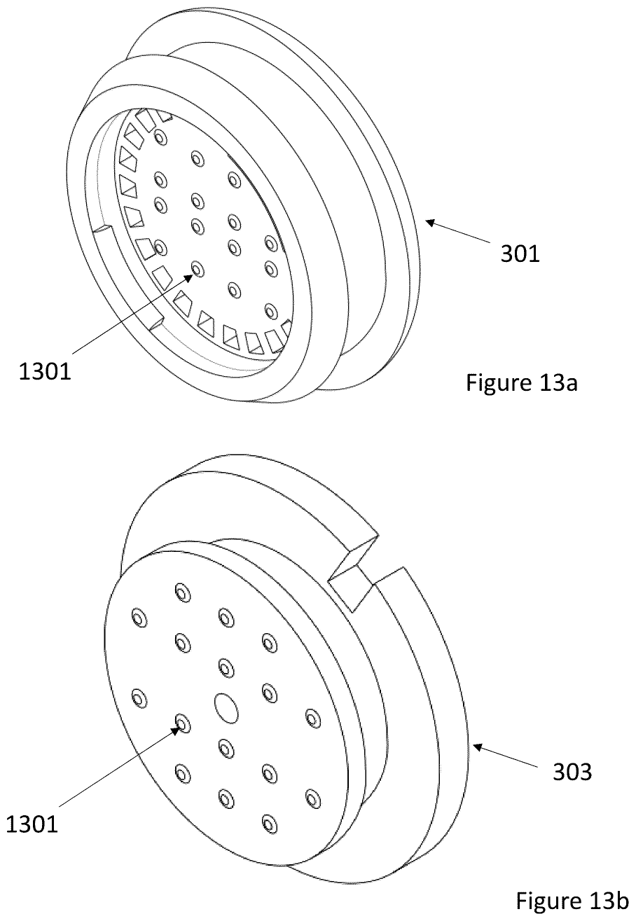

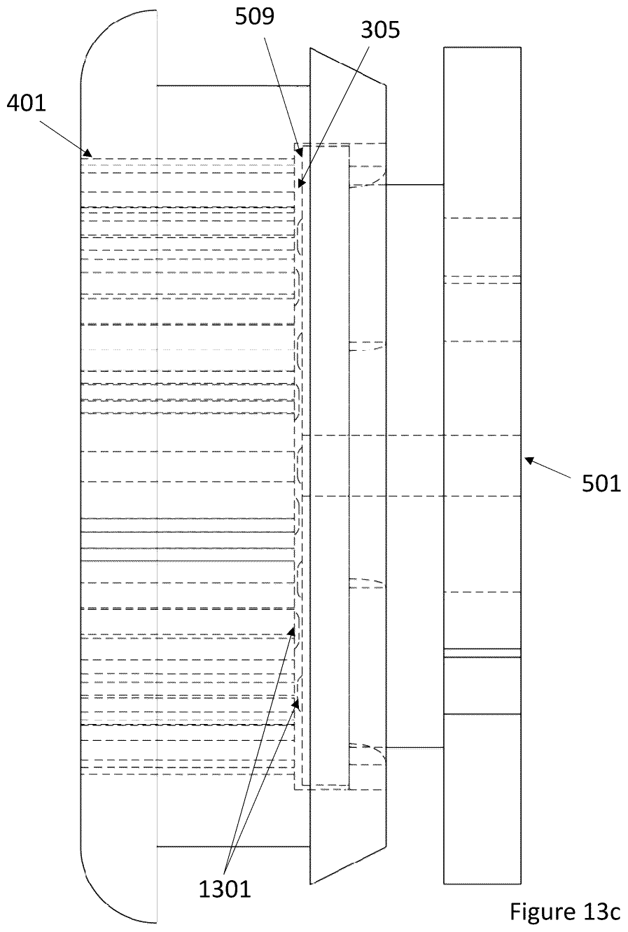

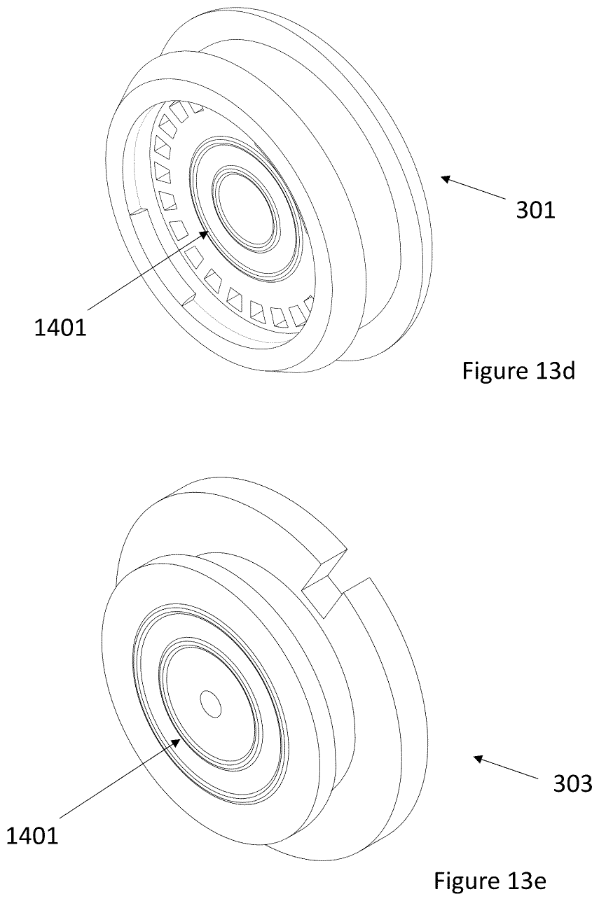

An example is shown in FIGS. 13a-c, in which the surfaces of the upper and lower modules that define the cavity 305 have a plurality of circular projections 1301. From FIG. 13c, it can be seen that the projections 1301 on opposing sides of the cavity 305 result in an undulating fluidic path between channels 401 and 501 in the cavity 305. Texturizing the surface in this way may narrow the cavity 305 to a width as small as about 30 μm in places.

FIGS. 13d and 13e show alternative texturization using circular ridges 1401 on the opposing surfaces of the upper 301 and lower 303 modules.

It will be appreciated that there are many possible options for texturizing the surfaces of the upper and lower modules.

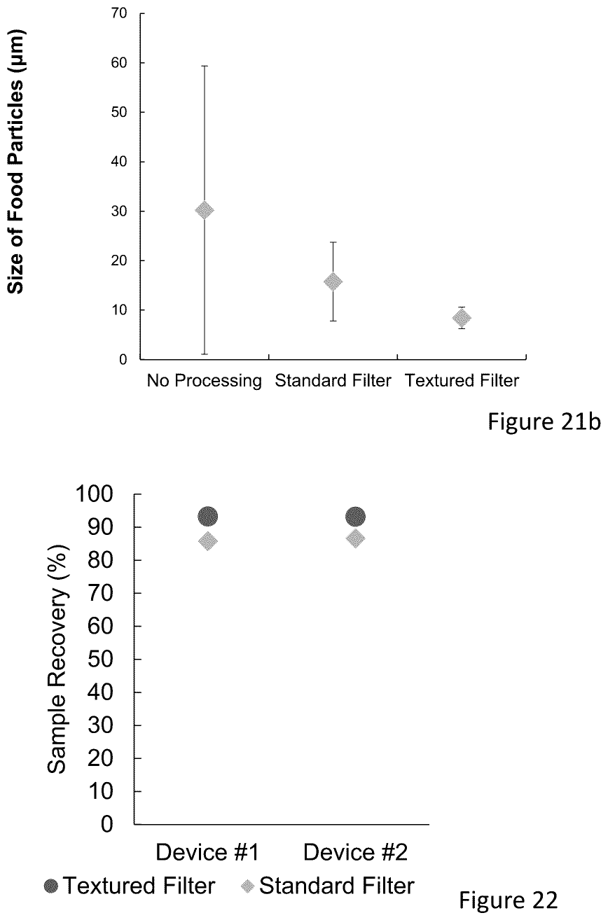

Textured surfaces may enhance the advantageous properties of the filter discussed above. For example, FIGS. 21a and 21b show experimental results for reducing the number and size of food particles in saliva, respectively, for both standard (i.e. a filter according to the embodiment of FIG. 3, without textured surfaces), as well as a filter having textured surfaces as shown in FIGS. 13d and 13e.

In order to obtain the results, about 1.038 g of chili flakes was suspended in 50 ml of MiliQ water and 2 ml of the suspension was processed through a device with a standard filter and a device with a textured filter. The samples were placed on a glass cover slip and imaged in six pre-determined positions at 4× magnification. The images were processed with ImageJ to automatically count the particles and their average size in each image frame (1150×1080 μm). The results are shown in FIGS. 21a and 21b as average±standard deviation. Both filters were found to reduce the number of food particles and the average size of the food particles relative to unprocessed saliva, with the textured filter providing the best performance.

The texturization of the filter was found to have no negative impact on the sample recovery achievable using the device of FIG. 1. FIG. 22 shows experimental results for devices according to FIG. 1 with both textured (indicated by circles) and standard (i.e. without texture; indicated by diamonds) filters. In order to obtain these results, back throat saliva was pooled from 12 individuals. Approximately 2 ml of the sample was processed through the devices with a standard and with a textured filter (two devices were employed in both cases). The weight of saliva collection devices with and without the sample as well as the weight of the collection tube before and after sample processing for each replicate were used to calculate sample recovery. In both cases, over about 85% of the sample was recovered.

Likewise, very little reduction in the protein concentration was detected experimentally using a textured filter compared with an untextured filter, as shown in FIG. 23b. In order to obtain these results, back throat saliva was pooled from 4 individuals. The sample was processed through a saliva collection device according to FIG. 1 with an untextured two-module filter according to FIG. 3 (denoted “GLOW standard processed” in FIG. 23b) and a textured two-module filter according to FIGS. 13a and 13b (denoted “GLOW Textured processed” in FIG. 23b). The protein content in samples that were unprocessed, processed with the device according to the two embodiments and processed by centrifuging (10 min, 300 rpg; denoted “Supernatant” in FIG. 23b) were compared using BCA assay performed as per manufacturer's instructions. The process was repeated three times for each tested sample. Not only was the recovered protein concentration for both devices according to the embodiments much greater than obtained by centrifuging, a slight increase in total protein concentration was detected relative to the unprocessed saliva in both cases, consistent with the results of FIG. 23a discussed above.

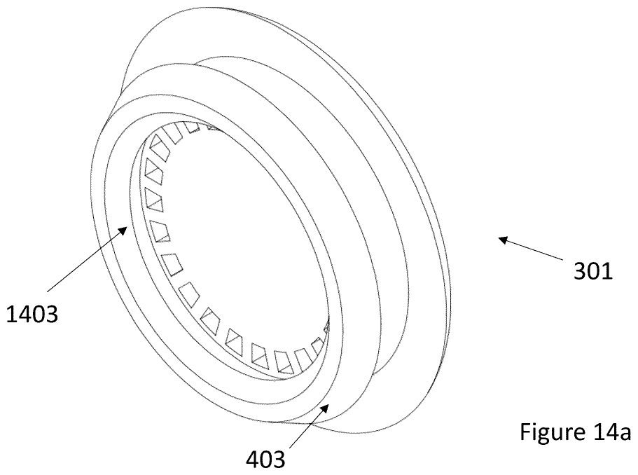

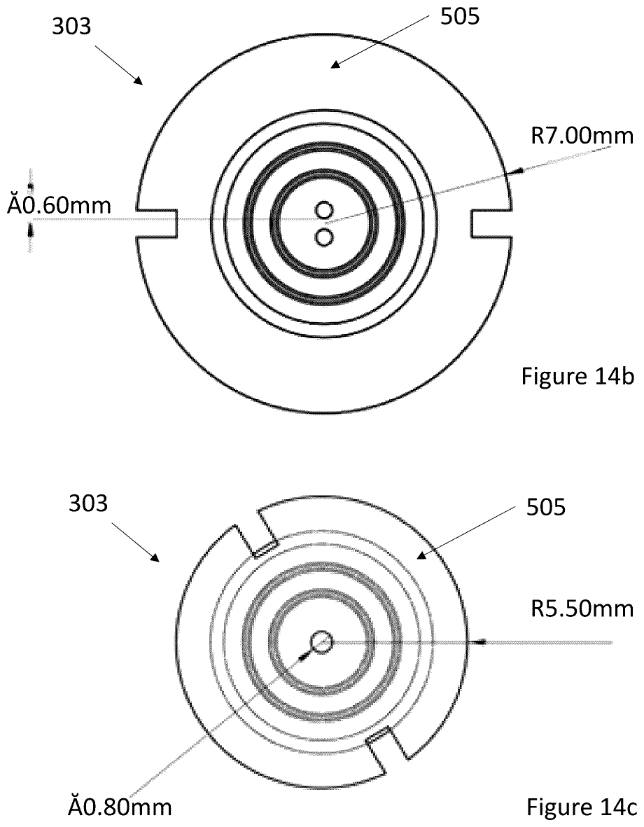

Further variations of the filter are also envisaged. For example, the projections 405 in the upper module 301 may be replaced by a single, continuous projection 1403 running around the circumference of the inside of the ridge 403, as shown in FIG. 14a. It will be appreciated that other configurations of projection could also be employed.

In further variations, the size of the shielding portion of the filter, in the form of the lower extending ridge 505 of the lower module 303 may be varied in order to shield the vent 211 or sides of the cap 105 from saliva to prevent release of the saliva from the collection tube 107. FIGS. 14b and 14c show lower modules with two different exemplary sizes for the ridge 505, giving rise to largest radii for the modules of about 7.00 mm and about 5.50 mm, respectively. It will be appreciated that other dimensions could be employed according to requirements and size constraints due to other components in the apparatus, such as the size of the cap 105. The ridge 505 could also be shaped so as to control the direction of saliva as required. The ridge could form an enlarged, flat or conical shield around the channel or channels 501 to direct the release of the processed sample towards the bottom of the collection tube 107 and shield the upper part of the tube 107 and the screw cap 105 from the sample.

Although in the preferred embodiment the cavity 305 is shown as being empty, an additional component may be inserted or formed (if, for example, the filter is fabricated using additive manufacturing techniques) within it. For example, an additional filter or bioactive substance in the form of, for example a film or a gel, for interacting with the sample could be present in the cavity 305.

Although the filter according to the preferred embodiment achieves viscosity reduction and/or increase in sample uniformity using channels which filter the saliva and induce shear, the channels may be omitted and viscosity reduction and/or increase in sample uniformity achieved without them. For example, the filter may comprise a multilayer metal mesh or a porous substrate configured to induce shear on the saliva and reduce its viscosity and/or increase its uniformity. Alternatively, the filter may comprise channels in addition to other features which induce shear such as a multilayer metal mesh or a porous substrate.

The plastic zip 113 could be replaced by any suitable sealing mechanism, or the funnel could have no sealing mechanism. In this case, the funnel 101 may not be divided into two separate sections 109 and 111, instead comprising only a single section. In this case, the rolling method of exerting pressure on the saliva sample as shown in FIG. 8 or squeezing the funnel using a clip, as discussed above, are preferred over the squeezing method shown in FIG. 7 as it reduces the chance of saliva being expelled from the funnel on the opposite side from the filter.

The apparatus 100 could be provided without a filter 103, the funnel operating as a pressure generator simply to drive the saliva into the collection tube 107.

Although the filter 103 is described as having a snap-fit connection to the cap 105 and the individual filter modules 301, 303, 3011 and 3013 are described as being snap-fitted together with respective snap-fitting elements, it will be appreciated that other mechanical attachment solutions could be employed in place of snap fitting, such as luer adaptors. The filter could also be configured to fit directly to the collection tube 107, via an adaptor or otherwise, without being mounted in a cap 105.

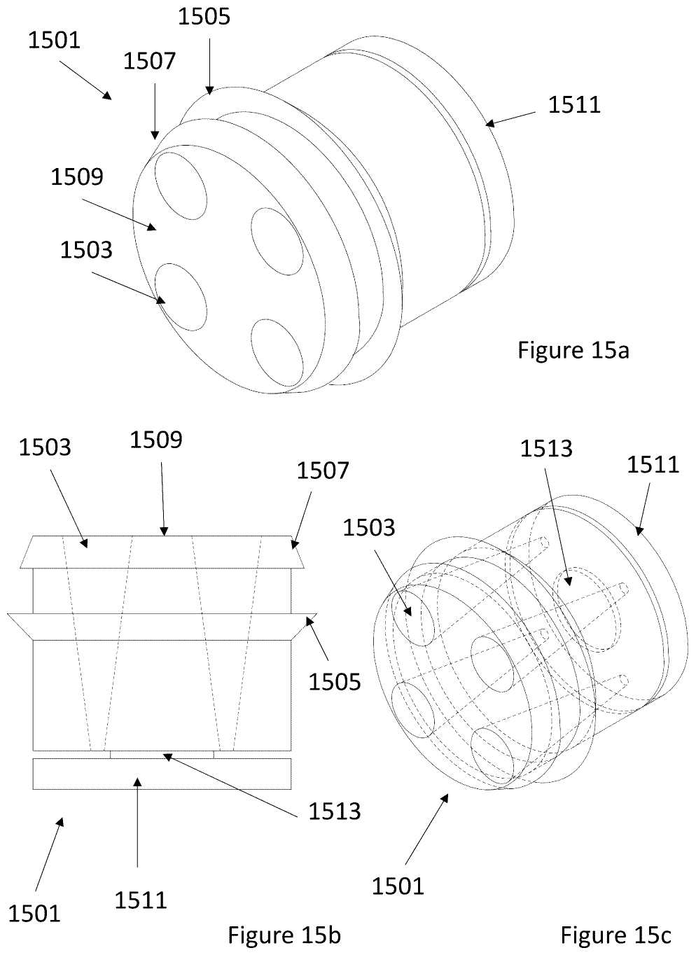

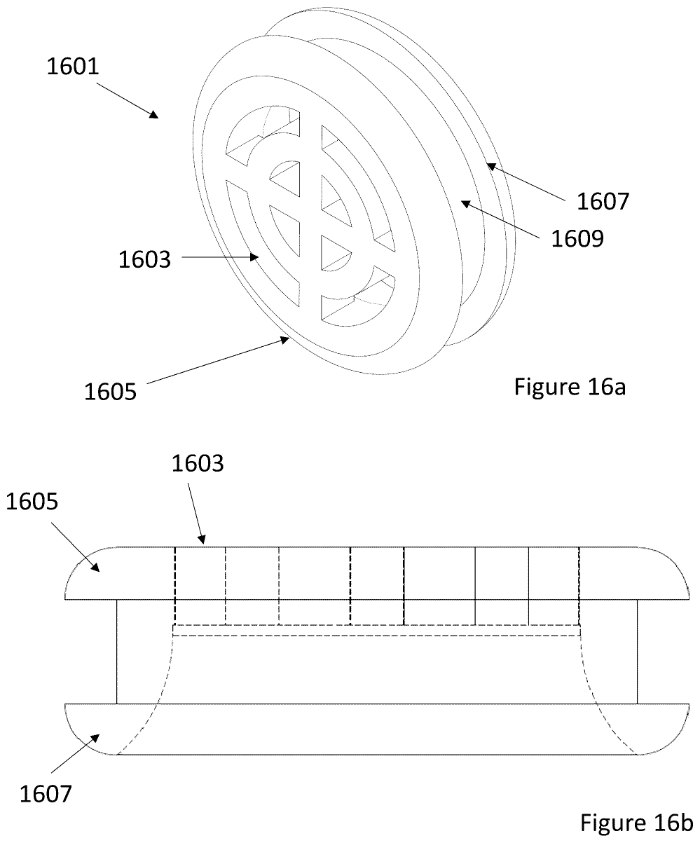

Filters according to two alternative embodiments will now be described with the aid of FIGS. 15 and 16. These filters are suitable for mounting in a cap 105 in the assembly of FIG. 1 in place of the filter 103.

FIGS. 15a, b and c show perspective, side, and further perspective views, respectively of a filter 1501 according to a first alternative embodiment FIGS. 15b and 15c show the internal structure of the filter. The filter consists of a single, integral module, which is broadly cylindrical in shape with ridges 1507 and 1505 to enable snap fitting to the cap 105.

The filter 1501 has four channels 1503 which narrow in cross section on moving away from the upper surface 1509 of the filter. The filter further comprises a flat hanging surface 1511 held in place below the channels 1503 by a pillar 1513.

The channels 1503 are configured to emit saliva directly onto the hanging surface. Shear stress is therefore imposed on the saliva by both the narrowing size of the channels and the pressurized contact of the saliva with the flat hanging surface 1511.

Using additive manufacturing techniques, such as 3D printing to produce filters according to the embodiment of FIG. 15, channels which are extremely small at their narrowest end may be achieved which may in turn give rise to high shearing rates and a significant reduction in viscosity and/or increase in sample uniformity.

FIGS. 16a and 16b show perspective and side views, respectively of a circular filter 1601 according to a second alternative embodiment of the invention. FIG. 16b shows the internal structure of the filter.

The filter comprises a single module with two parallel protrusions 1605 and 1607 to enable snap fitting to the cap 105. The modified screw cap 105 will have a hole of a diameter matching the diameter of the filter ring in the middle part 1609, smaller than the diameter at the protrusions 1605, 1607.

The filter has a plurality of channels 1603 arranged in approximately two concentric circles. The filter 1601 may prevent large particulates from entering the collection tube and also induce a low-level shear and decrease the number of small particles in the sample, such as spices. Food particles present in saliva are generally larger than about 0.80 mm across therefore a filter having channels with a channel opening smaller than this size may enable the majority of food particles to be removed from the sample. The size and shape of the channels 1603 can be altered, with smaller and fewer channels giving rise to a larger induced shear. The channels may also have a narrowing cross section in the direction of the fluidic path through the filter in order to increase the amount of shear induced on the saliva.

Although a compressive receptacle, in the form of a funnel, is employed as the pressure generator in the preferred embodiment, alternative components could be alternatively employed as pressure generators.

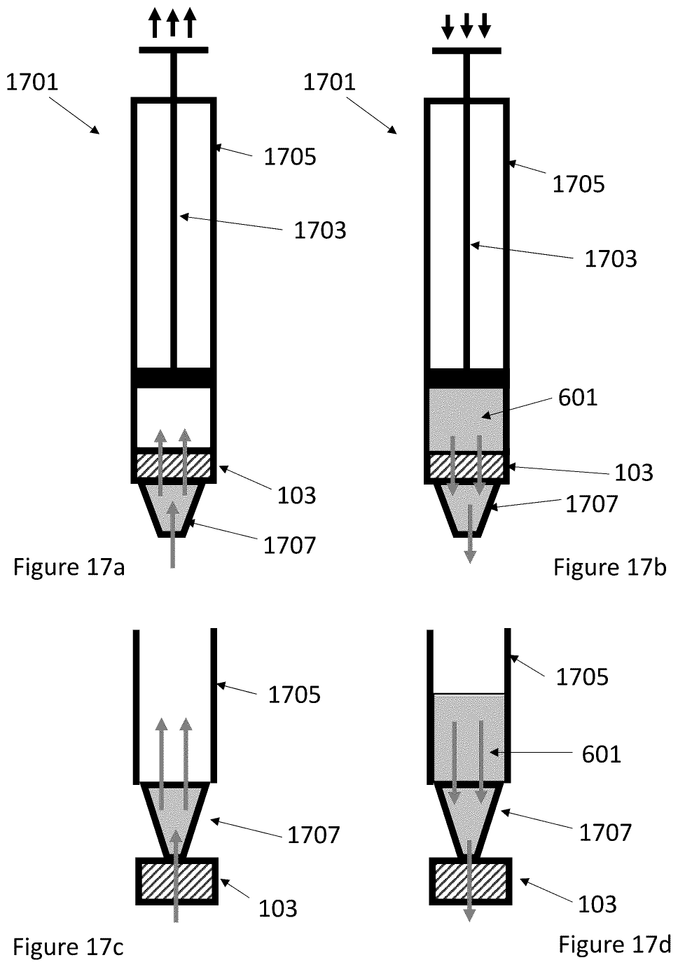

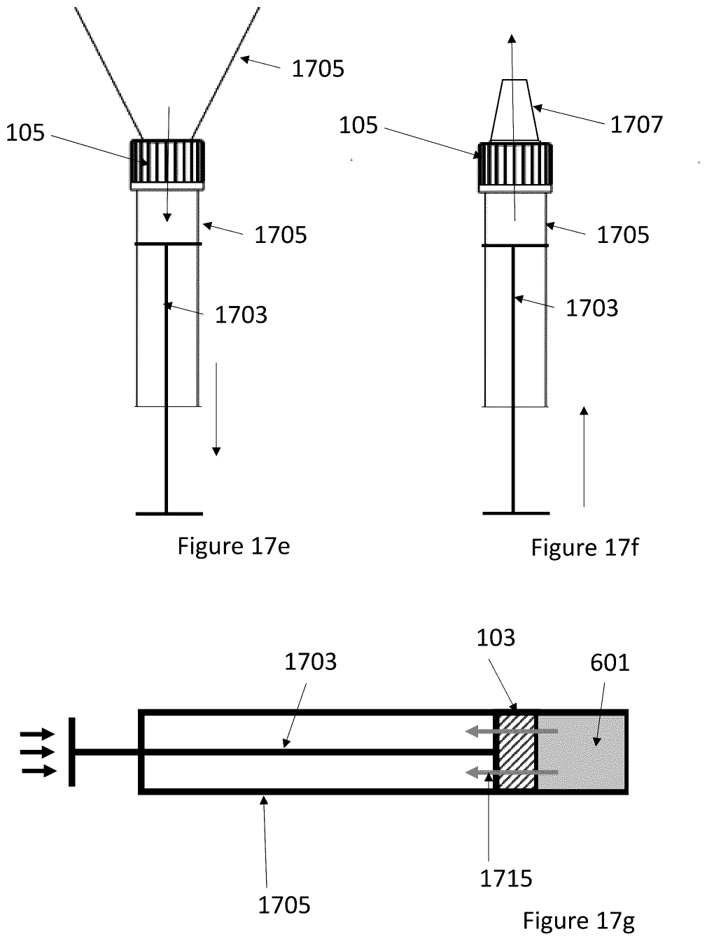

FIG. 17 shows a device 1701 according to an alternative embodiment in which a plunger 1703 is employed as a pressure generator for causing a sample 601 to be transferred though the filter 103. In the embodiment of FIGS. 17a and 17b, the filter 103 is located at the end of a vessel 1705. The vessel comprises a plunger 1703 which is withdrawn (as shown in FIG. 17a) in order to generate negative pressure in the vessel 1705 and draw a saliva sample from outside the device (for example, from a test tube or other receptacle into which an individual has expelled a sample) into the vessel 1705 via a luer adaptor 1707 and the filter 103. The plunger 1703 may then be pushed back into the vessel 1705 (as shown in FIG. 17b) to generate positive pressure on the saliva sample 601 and to drive it back out of the collection vessel 1705 and through the filter 103. Repeated cycles according to FIGS. 17a and 17b can be performed resulting in repeated shearing cycles of the sample 601 which may further reduce viscosity and/or increase sample uniformity.

FIGS. 17c and 17d show alternative arrangements for the device 1703 in which the filter 103 is attached to the narrower, output end of the luer adaptor 1707. Otherwise, the device 1701 functions exactly as described above.

The filter 103 could alternatively be incorporated into a cap 105 (as described above in relation to the preferred embodiment) for closing the vessel 1705 as shown in FIGS. 17e. In this variation, a funnel 1705 or other saliva receptacle may interface with the filter 103 in the cap 105 for receiving saliva from an individual and the plunger withdrawn to draw saliva from the receptacle in to the vessel, as shown in FIG. 17e. The funnel 1705 may then be optionally replaced with a luer adaptor 1707 for ejection of the sample 601 from the vessel 1705, as shown in FIG. 17f in order to achieve an additional cycle of shear.

In a further variation of this embodiment, shown in FIG. 17g, the filter is integral with plunger 1703 itself. As the plunger is actuated to exert pressure on the sample 601, the sample flows backwards through the filter 103 (indicated by the arrows 1715), thereby inducing shear.

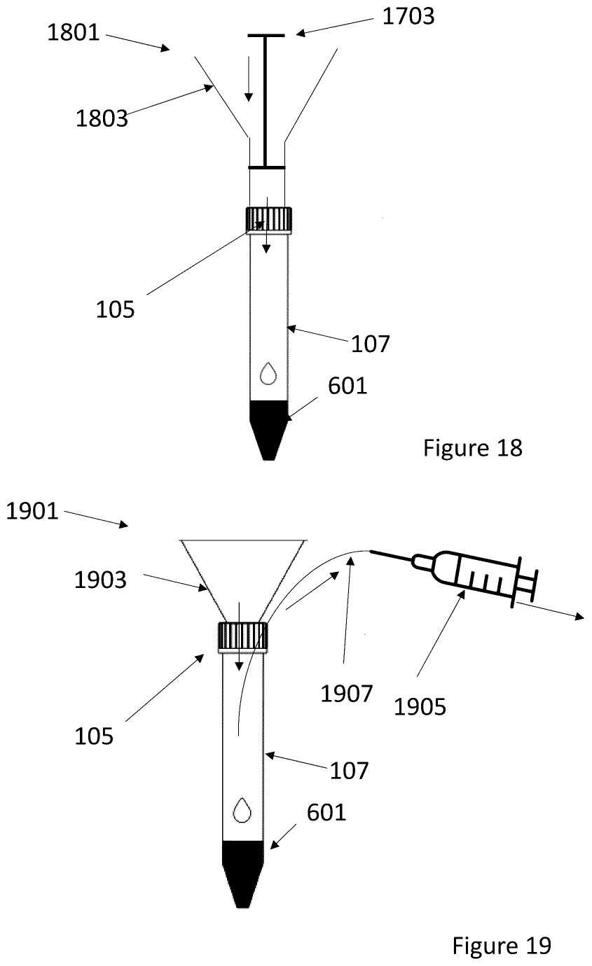

A further embodiment 1801 of the apparatus is shown in FIG. 18. In this embodiment, a collection vessel 107, cap 105 and filter 103 arrangement as in the embodiment of FIGS. 1a and 1b is employed. In this embodiment, however, the apparatus comprises a rigid receptacle 1803 for the saliva in the form of a funnel. Once saliva has been deposited into the funnel 1803, a plunger 1703 is inserted into the funnel as a pressure generator to exert positive pressure on the saliva sample 601 and drive it though the filter 103 located within the cap 107 and into the collection vessel 107.

Another embodiment 1901 of the apparatus is shown in FIG. 19. In this embodiment, a collection vessel 107, cap 105 and filter 103 arrangement as in the embodiment of FIGS. 1a and 1 b is employed. The apparatus further comprises a receptacle 1903 which may be rigid or non-rigid. In an example, the receptacle is in the form of a funnel. The pressure generator comprises a suction device 1905 in the form of a syringe which is arranged to suck air from the collection vessel 107 via a tube 1907 inserted through the cap 105. This withdrawal of air from the collection vessel 107 creates a negative pressure on the sample 601, drawing it through the filter.

In a variation of the embodiment of FIG. 19, the suction device could instead take the form of a sealed vacuum tube. After sample donation into a funnel, the seal of the vacuum tube could be pierced, for example, by screwing down a cap configured to do so onto the vacuum tube. The arrangement of the apparatus may be such that the release of the vacuum causes air to be sucked from the collection vessel 107 thereby causing a negative pressure on the sample to drawing it down through the filter.

It will be appreciated that various modifications and combinations of the above described pressure generators are possible. Although the pressure generators discussed above are described as being manually actuated, the pressure generation could alternatively be automated, in particular (but not limited to) variants in which a plunger is employed as the pressure generator.

Having now fully described the invention, it should be apparent to one of ordinary skill in the art that many modifications can be made hereto without departing from the scope as recited in the following claims.

The invention claimed is:

1. Apparatus for collecting a saliva sample, the apparatus comprising:
a filter; and
a pressure generator operable to generate pressure to cause the saliva sample to be transferred through the filter,
the filter being configured to reduce a viscosity of the saliva sample as the saliva sample is transferred through the filter.

2. (canceled)

3. Apparatus for collecting a saliva sample according to claim 1, wherein the pressure generator further comprises a receptacle being arranged to receive the saliva sample and being compressible to generate pressure to cause the saliva sample to be transferred out of the receptacle through the filter, the receptacle comprising comprises a seal operable to seal an upper, shielding portion of the receptacle from a lower portion of the receptacle, the lower portion of the receptacle being operable to undergo manual compression or the receptacle comprises a hydrophobic inner surface.

4. (canceled)

5. Apparatus for collecting a saliva sample according to claim 1, wherein the pressure generator further comprises a receptacle being arranged to receive the saliva sample and being compressible to generate pressure to cause the saliva sample to be transferred out of the receptacle through the filter, the receptacle being is in the form of a funnel.

6. Apparatus for collecting a saliva sample according to claim 5, the width of the funnel opening is in a range of 8 cm to 20 cm or the funnel comprises a plurality of handles.

7. (canceled)

8. Apparatus for collecting a saliva sample according to claim 1, the pressure generator further comprising a plunger operable to generate a positive pressure on the saliva sample to drive the saliva sample through the filter or to generate a negative pressure on the saliva sample to draw the saliva sample through the filter.

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. Apparatus for collecting a saliva sample according to claim 1, wherein the filter comprises a plurality of channels, a multilayer metal mesh and a porous substrate, and wherein one or more of the plurality of channels has a cross sectional width in the range 0.03 mm to 3 mm or wherein one or more of the plurality of channels has a narrowing cross-section in the direction of a fluidic path through the filter or wherein the plurality of channels comprises at least two channels having cross sections of different widths.

21. (canceled)

22. Apparatus for collecting a saliva sample according to claim 1, wherein the filter comprises a plurality of channels, a multi25 layer metal mesh and a porous substrate, and wherein one or more of the plurality of channels has a narrowing cross-section in the direction of a fluidic path through the filter, the filter further comprises a surface arranged to receive saliva output from the one or more of the plurality of channels having a narrowing cross-section.

23. Apparatus for collecting a saliva sample according to claim 22, wherein the surface is a hanging surface.

24. (canceled)

25. Apparatus for collecting a saliva sample according to claim 1, wherein the filter comprises one or more of a plurality of channels, a multi-layer metal mesh and a porous substrate, the filter comprises a plurality of first channels for receiving the saliva sample, a second channel and a third channel in fluidic connection with the plurality of first channels, the plurality of first channels being fluidically connected to the third channel by the second channel.

26. Apparatus for collecting a saliva sample according to claim 25, wherein the second channel has a narrower cross-sectional width than the first and third channels or wherein the second channel forms a non-zero angle with the first and third channels or wherein the second channel is substantially perpendicular to the first and third channels.

27. (canceled)

28. (canceled)

29. Apparatus for collecting a saliva sample according to claim 25, wherein the plurality of first channels is arranged in a substantially circular configuration or wherein the plurality of first channels is arranged in a plurality of substantially circular concentric configurations.

30. (canceled)

31. Apparatus for collecting a saliva sample according to claim 25, wherein the third channel is arranged substantially centrally in a lower surface of the filter, or wherein the filter comprises two third channels, or wherein one or more walls of the second channel has a textured surface.

32. (canceled)

33. (canceled)

34. Apparatus for collecting a saliva sample according to claim 25, wherein the filter comprises stacked first and second modules, the plurality of first channels being comprised within the first module, the third channel being comprised within the second module and the second channel being formed at an interface between the first and second modules.

35. Apparatus for collecting a saliva sample according to claim 34, wherein the first and second modules are connected via a first snap fitting.

36. Apparatus for collecting a saliva sample according to claim 34, wherein the filter further comprises, in the second channel, one or more of a further filter and a bioactive substance.

37. Apparatus for collecting a saliva sample according to claim 34, the filter comprising a plurality of alternately stacked first and second modules.

38. (canceled)

39. Apparatus for collecting a saliva sample according to claim 1, the apparatus further comprising a cap operable to close a collection vessel, the filter being comprised within the cap, or the apparatus further comprising a luer adaptor for connecting the filter to a collection vessel.

40. Apparatus for collecting a saliva sample according to claim 39, wherein the apparatus further comprises the cap operable to close the collection vessel and the filter is being comprised within the cap, the filter is connected to the cap via a snap fitting.

41. (canceled)

42. (canceled)

43. Apparatus for collecting a saliva sample, according to claim 39, wherein the apparatus further comprises the collection vessel, the collection vessel further comprises saliva detection and/or deactivation media, the filter being configured to prevent backflow out of the collection vessel.

44. (canceled)

45. (canceled)

46. (canceled)

47. A method of reducing the viscosity of a saliva sample, the method comprising:
applying pressure to the saliva sample to cause the saliva sample to be transferred through a filter, the filter being configured to reduce the viscosity of the saliva sample as the saliva sample is transferred through the filter,
wherein applying pressure to the saliva sample to cause the saliva sample to be transferred though the filter further comprises alternately applying positive and negative pressure to the saliva to cause the saliva sample to be transferred back and forth through the filter.

48. (canceled)

49. (canceled)

50. (canceled)

51. (canceled)

52. (canceled)

53. (canceled)

54. (canceled)
